SEQUENCE LISTING (57 sequences)

SEQ ID NO: 1 - protein, 238 aa, Dictyostelium discoideum
MRINFKLILIILTSFYGIINCQSTCPYSKTVINGASATFYSAMDNGNCGFGKLTGPTGPGNYMIAALGTKLYQNGAQCGQCFKISNSKNASVTVMATDSCNDAGYCQRDNHFDLSPTAFSILGAQSQGVLDGLSYVKVPCRVSGNVKVMLKDGSNAYWTSFLVFNNAIDVKQVSIKLSGSSTYVPLTQTTYNYWPSSITAGSFQVRIESIGGEFIYVTIPSVVSSKIYDTGSQFSSSC

SEQ ID NO: 2 - protein, 335 aa, Dictyostelium discoideum
MKFNTIFLVLSIVKFILISAQSCPFSQSIINGASATFYTAIDAGNCGFEKLNGPLGPGNYMIAALGSKLYQNGAQCGQCFKISNSKNASVTVMATDSCHDAGYCQRDNHFDLSPAAFSILGPQSQGVLDGLSYVKVPCEVSGNVKIMMKDGSNDFWTSFFVFNSKVIIKQVSIKLSNSNQFVPLSQTTYNYWPTSITGGQFHVRIESIGGEFIYVTIPKVESRKVYETSGQFSTSCSNLNENNPINYKPQTFNSQQTSNNQNSNTQTPTKQPSPNSQNFIPSYCQQYIQKPNYIFAKESKEMLVLNENENIESNSLKLLPNFLLLILIILLNINF

SEQ ID NO: 3 - protein, 286 aa, Dictyostelium discoideum
MKTFVLFVILLCLTFLSISKSETCPFSQSLVSGASATYYTDPNAGNCGYENLMGPLGPGNLFIAALGPNLYNNGKNCGQCFNISSPYTNRSVVIMATDSCPDSGYCQRSSHFDLSTQAFDVLGAQSIGVLEGLTYYKVPCGVNGNVKIMMKDGSNDYWTAFLIYNSKVTIKDVSVKITGKSTYTSLTQSSYNYWISPNMVPGSFDVRIESVGGEFIYITIPKVESRKQYETSSQFSVDGCVGTPSGPSGGLGSPSTGASIGTPSDASSLTLYALFSLTILFLVMLN

SEQ ID NO: 4 - protein, 352 aa, Neosartorya fischeri
MLYQRLTTLGVAALVAASSVSASPMVGGAKSRCRAGYNKAVSHVPTTHEATPSINPPVELSEAPSQSPWPTVDSPSAASIIPTVDEITHVKKPQADEDPDASSSSSSSSTTSALPSSPATQQQDTVPAAPAEPATAASQTNNKAAAAASSSSSTTHSGKATFYGGNISGGTCSFTGYTLPSGLFGTAYSGAAWNNAAECGACVSVKGPNGKTIKAMIVDQCPECEQDHLDLFQDAFTQLADVSKGIIPITWSFVPCGITSPVVLKNKEGTSRYWFSMQVMNANEPVAKLEVSTDGGKTWQGTTRTSYNFFEESSGFGQDTVDVRVTGQSGATLTVKNVGTSSGSSVTAKSNL

SEQ ID NO: 5 - protein, 344 aa, Aspergillus fumigatus
MLYQRLTALGVAALVAASSVSASPMARGVKGRCRAKYNKAVSHVPTTHEATPTINLPVELPETQSQSPWPTVDEIVPVKKPQADEDPNASSSSSSSSSSLSTTSALPSSPATQEQDTVSAAPAEPTTAAPETVNKAATTASSSSSTTHSGKATFYGGNVSGGTCSFTGYTLPSGLFGTAYSGAAWNNAAECGACVSVTGPNGKTIKAMIVDQCPECEQDHLDLFQNAFTQLADVSKGIIPITWSFVPCGITTPLVLKNKEGTSPYWFSMQVMNANEPVAKLEVSTDGGKTWQGTTRTSYNFFEKSSGFGKDTVDVRVTGQSGATLTVTNVGTSSGSSVTAKSNL

SEQ ID NO: 6 - protein, 428 aa, Aspergillus clavatus
MRYQRLTTLGVAALGAAASVSASPLFQRAENQCPPGYTMSVYYVTVTASPTPSIEPVTTLSSSSTTTVTTTVTPEAPAKTSSSLDVESSVPERTPVETPVASEPPVVPSSSSTKQIVVPTAEPVPVPVPEPIEPAPSSTKTTVATEPHTTAAPPVVPSSSSTKQIVIPTAEPVPVPVREPIEPAPSSTRTTVATEPHTTAAPPVAVPPATAPLAATTTAAPPPPASRPAGSNSGKATFYGGNVSGGTCSFSGYKLPAGLFGTALSKARWSDAAECGACVSVTGPNGNSIKAMIVDQCPECESNHLDLFQDAFAELADISKGIIGIDWSYVPCEIDSPLVLKNKEGTSRYWFSMQVVNANEPVASLEVSTNGGSTWQPTTRTYYNFFENASGFGADTVDVRVTGVSGKSLTVKNVGVGSSSSVTASSNL

SEQ ID NO: 7 - protein, 420 aa, Aspergillus oryzae
MKYQRLASLGLAALSVTGSVSASPLIRHEGESVCPSGYTQSVYYVTVTASSTPASTSSVEPTTTIESTSTVTETTVITPEIPAQSPTSTPVEIPAPVETPAPVETPSPAETPAPVETNTPVEPTTSSSTTETPVVAPTIATPSTADVQPTEVVAEPSTSSSSTEEPTATPIAAETPSTTVDAQPTTAAAAPTTKQLKLSTTSTAAPAASVTSSSTGSSSSSSSSSESNSGEATFYGGNLSGGACSFTGYTLPSNLFGTALGSPRWDNAAECGACVAVTGPNGNTIKAMIVDKCPECDSNHLDLFQSAFTELADISKGVIDITWNYVSCDIDTPLKLKNKEGTSAYWFSMQVVNANEAVTSLEVSTDGGSTWQSTTRSDYNYFENSSGFGTATVDVRVTGKSGKVVTVNNVSVSSGVEVTA

SEQ ID NO: 8 - protein, 341 aa, Aspergillus terreus
MSVYYKTITLEPTPSVEPTTTIQATSTVTETVTAIPESTLTSVSVPMETPTTGANVAPTESPVDAIETSVSVGLPPSSSTSTSATVHQKVIVPAESTTTVAPQPTTAAVVVSTSQAQQPTTTAAAPSASTSASSSSSSTSKSETYSGEATFYGGNVSGGTCSFTGYTLPSGLFGTAYSGAQWNDAAQCGACVQVTGPSGNSIKAMIVDQCPECEATHLDLFQNGFSELAALSEGIISIDWSFVSCDIDTPLVLKNKEGTSAYWFSMQVVNSNEPVTALEVSTDGGSTWHATTRSFYNYFENDSGFGTDTVDVRITGQSGKTVRVNNVGCSSGSSTTASTNF

SEQ ID NO: 9 - protein, 378 aa, Penicillium chrysogenum
MKYLRLASVAALFSAATVSAGPLGAREHDGYCPKGYTMSVYYKTITVESYPSTTSVESTPAIVESQPPAEPTLASSSPAIAVESTTPVAVIQVETTSSSAPAAETEVAETAAPKTDAVVAPLPTASAETAAVIETIAAPAVKTPVVQPSTTEAPAEVASTSTKSSTSTSSTKSASTGSSNGTPGKATFYGGNVGGGTCSFSGYTLPSHLFGTALSLQRWDDAANCGACVSVTGPKGNSIKAMIVDQCPECESNHLDLFQEAFAELSDISAGIIQTTWSYVPCDLDGPLKLKNKEGTSAYWFSMQVVNANEAVTALEVSTDGGSSWQSTTRTYYNYFENTAGFGTSTVDVRITGASGSTVVVKDVGVSSGSEVTAGSNL

SEQ ID NO: 10 - protein, 524 aa, Xanthomonas campestris
MKYRCLASLGIAALGAAATVSANPLLNREAEGQCPQGYTQSVYYKTITLQPSSTASTAPTSTLASAGEGQCPVGYTQSVYYKTIPLQPTSTASVEPTSAPAVEPTSTPAVESSSTSIDQVTTLHSSSTVIVTEVVTATPAVATTSSAVESAAPVATTPSAAEASPAAETSTAVEATSSSSSSVVEVTTNAAANAAATTAAAQPTPEAVTTEVATTEAAATTEAAAQPSPEAATTEAVTTQAAAAAATTEAATTQEAAAAATTEAVVQSTAEAATTEAATTEAAAATQAAATTVESKSTSTSSTAASAATSSASSSSSSTSSALSSEYSGEATFYGGNVSGGTCSFTEYTIPSGLFGTALSSQRWDNAAECGACVEVTGPSGTKIKAMVVDECPECDSNHLDLFESAFSELADISKGVISIDWEYVACGITSPIELVNKSGTSAYWFAMQVVNSNLPVTKLEVSTDSGSTWQSTTRSSYNYFENQSGFGTDTVDVRVTAEGGSQITVKNVSVSSGSSVTASSNFE

SEQ ID NO: 11 - protein, 366 aa, Emericella nidulans
MKYQHFSSIALAALSASTFVSAAPLAPEENGSCPAGYSPSVYYITVTAEPSSTVRPTSSAPISSTPTSTSTSTSTSETSLTTLASTSTGDVTVTSSSTAGLIETIPAVVVNAATSTTSESATSTSALSISETAPTQVAVARPSTTTAAEKTSSTSTSSSSSSTSTNSGATTGEATFYGGNLSGGTCSFTDYTLPSHLSGVAFSGQAWDNAAECGACIAVTGPNGNTVKVMVVDKCPECAQTHLDLFESAFTTLASASEGQIPISYSITPCGITSPLILRNKSGTSAYWFSMQVVNANGAVKSLEVSTDNGSTWQETTRSDYNFFENSSGFGTDTVDVRVTGVDGGVITVKNVSVASGESVTADGNF

SEQ ID NO: 12 - protein, 223 aa, Magnaporthe grisea
MASHSAIITRLLVLILAITVRQTAAVNGKASHYGGNLSGGNCMLTSYTIPAGTFGTAIAISNYDNSNMCGVCLNVKGPSGSMKVMVVDSCPDCPPNKLDLFENGFSNIGDFNAGIVDVDWEVTPCDITEPLQVRNKDGTSKYWFSMQVLNSNQQVQSLEVSTDGGATWQGTTRRPYNFFEKTSGGGFGADSLTVRVTCAGGGQVTMQNVGVAGGSSFTASGNC

SEQ ID NO: 13 - protein, 397 aa, Pyrenophora tritici-repentis
MKGAIFSILPFVGLAFAQESACPAEPDETITETEVETVYKTATPTPALVADATTSPGSCGPSIVSVTMTEKVYVTVTYTSGTYVTPDAVKTIDVTSTSTLDIYTTVTLHPSGSVHPSGGYFSNSTTVALFKPSQLLSSAPATHTPIPPPRPTTTMSTIAALKPTSSTPSPAAATSTSASPSATAATVAPPTTQTKAGTTKRGSATWYGGNLSGGTCSFVGYTIPAGIYGTAISDFNWDTAGACGTCVSVTGPKGNTVKAMVVDQCPGCGPNHLDLFPDGFAALASPNAGNITVDWAVVPCGISSPIVLKNKSGTSKFWFSMQVMNANVGVAKLEVSTDGGASWKQTKRQPYNFFENPAGFGTDAVDVRVTSTDGKSIVVKNVGIKSESKTTAGGNFA

SEQ ID NO: 14 - protein, 397 aa, Phaeosphaeria nodorum
MRSIIFALLPLAGLAFAESSTCDAQRKTVTKTQYEYQTVTPVLAEKGRATPEAVTSSSCSRRTVTATKVEKVTVTVGASNTDPGTIDVTSISYKTVKTTVTVRPSGAPPAASHASSSLQWGNYPNATYHASSSARSTFLTSSSAPATPTSDLPSFSVIGQAIPTSEAAPAAAATPKVAAAAPQEAPETVPGTSSAAAGSKRGEATFYGGNTAGGMCSFTGYTIPAGLFGTALTDSDWDSGNACGQCVSVTGPDGKTKITAMVVDQCPGCGPHHVDLYPDAFAKLADPSKGIINVSWDFVPCGITTPIVLKNKSGTSKFWFSIQVMNANVGVSKLEVSTDGGASWKATTRKPYNFFEQPSGFGTDSVDIKITSIDGKTVVVKGVSVTPNALKTAGGNF

SEQ ID NO: 15 - protein, 304 aa, Sclerotinia sclerotiorum
MVFSIKNVAIASAFLSVAQGAVMGKRALSGQATSYGGNTNGGACSFSTYTLPSNILGTALSDSNWATAANCGRCVSVTGPSGNNITAMITDECPGCGTNHLDLYQNAFTKLAALSVGIIDITWDYVPCPITTPLQVHLKEGVSAYWFSMQVVNAAEGVSKLEVSTDGSKTWKSTTRTTYNFFENSSGFGTTSVDVRVTGLSGSTVIIKNVAVTPGLVVTASGNLKSGSTGSAKTVVPATVSSTSSEVDTVEPVVAADAVATPSASPVAAVSTPDVVATPASSPSPSVAEEPTPESEESDECDES

SEQ ID NO: 16 - protein, 293 aa, Frankia sp.
MSPAPGFVSPVRRALLGMAVAAATAVLATGCQPAVGGGPPEPDPVSTATAASTPGPSSTPTGPSAPPGRPSVTGAPAPGAPAGPTTVPVTPGRIQPGVVRTGPATHYGADGGGNCMFDRLTDPAMPVVAMNELDYETARACGAYIEVTGPGGTTVVKVTDRCPECGPGHLDLSQQAFARIAGGVPGLVDVTWRLVSPADIGSVQFRVKEGSSAYWLAIQVRNHRNPVVSLEVRVNGAWTALPREMWNYFVAPQGLGPGPFTVRITDVYGEQLVETVNLAPASVQTTGSQFARH

SEQ ID NO: 17 - protein, 302 aa, Streptomyces sampsonii
MVGSTAGLVVTGLLVCLAIAMQPDRKDDAGRAAATAVADSQETGPASSPTGSKSPTPSPSAVSASLKAKAKKPKASASPSATKPSSASPRKATTTSSLAGRILGKVTYPGVATVYKAGVGDGACSYGPSSDMMIAAMNTTDYETSKACGAYVFVRAANGNSVTVRITNECPLPCAPGQLDLSEQAFAKLAPVSTGRLAVTWSLVSPSSVGTISIRYKIGSSPYWCGIQAIGHRNPLARLEVSTGGGWRQLARTDYNYFLSPDGSGCGKAIRLTDIYGEQLTVNGVALRPDVVQSTGLQFARH

SEQ ID NO: 18 - protein, 346 aa, Sorangium cellulosum
MKLGSMPRGISWPLVSLIALGVAGCSDGGGGDAGSAESASSAAGGPGGAGAVGASASAGTGAGGGDGVTTGAGAGPGSSTTAGSTSSTSGGGGDPSATSTGASTSTGTGSQPVSCDYPAEYANGSITFYTLDMGSTEVNCSYPIVGRNPDVVGHVPFGGGQYFAAMNTADYNAAAMCGACVEVSRDDGRKKVQAMVVDQCPIATNPKCKAGHIDLSKNAFLQIGEEREGYLGTTNGGAAGKISWRYIPCPTTSAVSFRLKDASNKYWNEILVEGHAHPIEKLEVEINGTWQTATRQSYNYFTVGDGNMGNAPYHVRATDINGSTVEAMLELKAGNQPASGQFAACN

SEQ ID NO: 19 - protein, 438 aa, Sorangium cellulosum
MRLGAYRSAVALSFLGLVAAACTVATGGEEEADALYGAVVTTDGLTPAALTMPSDWGSGYCANVIVANDGTAPVTSWTVVINLNQAAVSSWWNAASSQSGSTLTVSPRSGSPSIPVGSSVSFGFCANATGSNYRPTLVSVTKTGGGSSSSSSSSSSSSSSSSSSSSSSSSAGSTGSAGGAGGAGGAGGSGGSGGSTGAGGSSATCSIPLPSYTSGNGSATWYSLDQGTAQVNCSLPIQQRNPDSIGHVATGGGRYFAALNTADYNTAAACGACVEVSRDDGRKVVATVVDQCPTASNPKCKAGHIDLSKEAFAQIGALDEGYLGTGNGGVRGKISWKYVPCPVEENVSIVLKEPGNAWWNELRVQGHRTPVRKLEVYVGGRWTEATRQPYNYWRVGSGNMGQGPWRVRVTDVLGKTIETSINTTTAEQLTSSQFPVCQ

SEQ ID NO: 20 - protein, 243 aa, Stigmatella aurantiaca
MRFGKAFSSHALLVAGVLALTACSDNGDGDGDGGGGGGGGGGGGGDVGLGKDPKDGRVNAYETEGDGSCSFGPPVGEKFVAALSSTEFLGAAVCGACAEVSGPNGKVVARVVDICDSCAPDQMLLSQEAYSKVASGGEVKMSWKLVACSVEGPIRYHIKDGSGPYSYFAIQVRNHKVPIKSLEVMRGSNWETLTRENYNYFVGNALGFPPLQVRVTGTTGEVLEDTLNSIDGDALQPGKAQFK

SEQ ID NO: 21 - protein, 298 aa, Plesiocystis pacifica
MSPARSIALLSLTLVSLFGCAEEPSEEGSTGGATLGFPDTGDEADTTTAGESSEGEASSGESGSGESSGETSGEASSGDTASETSGGQGSCGPAQVFEGQATWYELATDLVNCSYPTGTLPQFYGALNTAQYADAAMCGACARVTGPKGSVDIQIVDQCPIATNPICYEGHIDLNPPAFEQIGEIIDGIIPITWELISCDDPGTIAYSFKEGSSQWWTGVLVRNHRNPIAKFEYDTGGGNFKAVPREGYNFFVDPDGFGTGPFTFRVTDIHGHVITDVDIPLQLGDPVVGGSQFPACD

SEQ ID NO: 22 - protein, 243 aa, Stigmatella aurantiaca
MRALPLVSLRHLTAVGMLGLTACSSSEPDDSGFTNDPEGEVRALGEFRQGIATWYNATGEGHCGYDASPKDMDVAAMNAPQFANSAVCGSCAEVEGPKGTVRVRIVDSCPECGPGHLDLSEQAFAKIAAVADGRVQTRWRPVTCAVSGPVRYRFKEGSSQWWTAIQVRNHRLPIQKLEWQRENGAWVDMQRQDYNYFLASPGVDTATTKLRVTASDGQVLEDTLPRVEEKKVFDGAEQFSLKK

SEQ ID NO: 23 - protein, 233 aa, Myxococcus xanthus
MAMHPLRSKSRRFLLPVAAVFVACGGCSDDPSGGGAPLGEEQQGIATFYNATGSGNCSYEPTGDLMVAAMNTPQYANSAACGQCVDITGPKGSVRVRIVDRCPECESGHLDLSREAFARIAEMQQGRVNITWTPVSCDVAGNIEYHFKNGSNPWWTAIQVRNHRLPIQKLEWRRGTGGWQDVPRESYNYFVNLSGMGDGPFSVRVTAVDGQQLEDTLDRVLDNRSAEGSGQFR

SEQ ID NO: 24 - protein, 254 aa, Leptothrix cholodnii
MRRHPRRFPHRMLDLESALKRLACLCTLAGLAACGGGSPLAAASPTVDTDGGGTGATLGATRQGEGTYYAATGAGACSYDASADRMVAAMNHTDYAGSAACGEHVRVTGPLGTVTVRIVDECPECAPGDVDLSAEAFARIAEPVAGRVPITWQVVTGAVSGPVQYRYKEGSTRYWTAIQVRNHPVPIARLEIQPDGTGNWIAVPRTDYNYFVHPVAIGAGALQVRVTSSTGVALIDRLPQPQGGLVVDGTGQFQ

SEQ ID NO: 25 - protein, 1512 aa, Roseiflexus sp.
MPARFRFLRYVLIVVLLVVSIPLLSAPGHSSFIERMTTPPAAIAQSAAPVGPQPGDVFRQFQDDLNSRQTSVCNSSPANCGTIPSEQQRQARNVDLANAVRAEAVFEYWGGHLASTQQEFNVNGGPWTPYPDIQGIPPDTLPTCYFRAPTNAIAPLPLSGTGGVRPPGSTGSTVTVNYRYRIGPQRSLPQCASGTNFYWGNMWIYEVTTRIYYNSTIPRPDGAITSPTDGATITENRSANQFPRITVRAQASTGRSIARVDLIGNYLDYDWDGDGILREWHYALRHGVWERTIGTSTSPSRGTATSGEYDFTWNTEWIPDQDQPVQLMARITDNTGVSYMTEAVTVTFNRVGRSVRMYTTWSDYYGQRVFGPPRNFGFNTYSQPRIGKTAFISIPDPLTNATRARVILYSWSGRSSTALSTTVAINAFSVWSGNSFGIFHDYNFDTRDLNNPRDFLNQGDNILRIWSNRTDHALEVLWPGPAIFVEYSAPPETPVLRAIDTSFSTPEDQSLSFTLPVSNQARAALQYTILTPPALGTLRGIAPNLTYIPNPDVNGSDSFVFRVDDQYGRSATATVAINIQAVDDPPEITLGGGALLNPDDVLDRSGRVRDPDSSSLTATVDYGDGRGEQPLSLGADGSFRLIYRYRNGGRFPLTVRVSDGAQTSVATVLVTVITPGTPPPPGDWSLTGWRYRVPLEARSGAYPRVDHVAEFDVNFTTALSQAGGSGALLRESIRVVEVDAGGNVIDPNVVYQFDPVSAYNPTTNARGTLLVLLRGRTEANTTRRFYVYFETQGRFAPPTFTPLTVVVTPTLIYQRDLATVIETRTSDGQRNATFYYSLNGGALISMFDRSNADWISYNPAADSAGAYRGIPNVGPVFHPGYYNVFPIPSASGPLQGNEPGTNQRSSTTIEREGTLRTIVRSVSSNGQWEAVWEFFPNSVRMLVLPREPRGSNPFLYWWLYEGTPGGTLNEGDRWVRGDGLSGIAVDPSFSFNERFHADINGPEWVYFRAGEPSQGGRALWLAHHDDTDTTIEMYRYQNDSSGWQNGMTVFGFGRREWDGSSQTLRPELRNQRRFSLGFIDNAPLTFDSAVGREIEGRYRDLAATVGRGERLAPNLAPWAPGETYSVRPGQTLNVPAPGVRANDSDGDGDPFQVTLTGEPQNGTLTFNSDGSFIYVPNEGFVGADVFTYTASDGRLSAPPTTVTILVSPNQPPTARGSSATTTNLRPLTVPAPGVLEGASDPDGDPLTAVLISPPAGGTLNLNPDGSYTYTPRSDFIGEDVFQIAASDGEAQSAPVEVRITVRKENRPPTARGSSATTTNLRPLTVPAPGVLEGASDPDGDPLTAVLISPPAGGTLSLNPDGSYTYTPRSDFIGEDVFQIAASDGEAQSAPVEVRITVRKENRPPTARGSSATTTNLRPLTVPAPGVLEGASDPDGDPLTAVLISSPAGGTLSLNPDGSYTYTPRSDFIGEDVFQIAASDGEAQSAPVEVRITVTREVASTPEYRVFLPLLVR

SEQ ID NO: 26 - protein, 237 aa, Roseiflexus castenholzii
MPLRILAFAGVCLLLAGIIPVVRAQNDTHTVYLPLVVRDAQYRTGEGTYYAADGTGNCMFDPSPHDLMVAAMNHIDYDNAALCGAFIEVIGPKGSVTVRIVDRCPECARGDVDMSPQAFERIADLSAGRVPIRWRIVSPNLAGPIAYRFKEGSNQWWTAVQIRNHRNPVARFEYLRADGQWQTVPRQMYNYFVASSGMGPGPYTFRVTDMYGNVLTDTDIPFVEGGVINGAAQFPPP

SEQ ID NO: 27 - protein, 258 aa, Chloroflexus aurantiacus
MQWSAGRLQIALVICLTGFFWLIANPVFTQPPYRVHLPLVVRPAQNPLLSGEATFYLEADGSGSCLFDPIPGDKMVAAISYLNYGNADYCGAYVEVFGPQGSVVVRIVDMCPDNPGCGQNHLDLSPEAFDRIAPRAWGRVPITWRVISPPLSGPVQFHLKDGSNPWWLAFQVRHHRNPIARMEYRTPQGQWVQLNRTTYNYFIRQCQCANGELGPFTLRITDIYGNVLTETVQFTTLSRSSNSPGELVSGTGQFPYGP

SEQ ID NO: 28 - protein, 477 aa, Herpetosiphon aurantiacus
MAWRKGTFFSFGLILSLLLTSALFVAQGSAQRMQTIADPWALRSGRATFYDPTVGMGNCSLPVPSDMLLAAMNTTDYGLADYCGAYVTVNGPRGSVTVKIIDRCPGCVVGGIDLSPQAFERIAALEAGNVPITWQLISAPNVSGNVIYNYKEGSSQWWAGVQVRNHRNAIAKFEYRNPQGIFQNVDRVQYNYFLVPGGMGTGPFTFRLTDVYGNVFTDNTIPLRSEGDVAGNQQFPFVPTPGSTPTASPTRTASPTVVSPTTRPDQTIMNASSTEAGGSTHYALDGNLNTRWSSGLPQAAGQWIYIGLPRVTPISGIKLDAGSSGGDYPAGFIVQTRDDTSDWATVATGSGSSQITTINFAARNARYVRIELTARSANWWSIHELTVIFAAQPATLTPTILPPTNTIVPATVTPTLVPTMAPPPLTATPTLSAWQAYTNYSVGSLVQHNGINYRCIQAHTSLPGWEPQIVPALWQPL

SEQ ID NO: 29 - protein, 273 aa, Herpetosiphon aurantiacus
MLVRTHAVRWLLFVSLFMVIFGSYAWQQQSFAQNPATITNPWAIRQGQATYYTATGLGNCSIPVPSDLLFGAMNNPDYATADYCGAFVKITGPLGSVTVQITDRCPECQTGHIDLSPQAFDRIANRVTGIVPITWQLISNPATTGKVSYHFKDGSSQWWTAVQPRNHRNAIAKFEYRMGQAAYKVAPRFIFNYFIAEAGMGTGPYSFRTTDVYGNMIVDENIVLGDDVIRTGTQQFPYMAPPGSATPTPSPTPRPTIPPESLTEHVWLPYTRK

SEQ ID NO: 30 - protein, 243 aa, Hahella chejuensis
MSKLTFLIIGSILLLLCVRASYAENRVSATHTSAALHEGEGTYYFYNGGGHCSVPVPAMFTAAMNQTDYNGSQACGGCVKVTNRNNGKSVVARVDDSCPGCNPGDVDLTDAAFAQISPLEAGRIPISWDYVPCDYPSVLLYFMEGSSQWWTAVQVREQRYPVSSLAYRESGSTGSYQEIAREDYNYFVERSGMGTGPFDFRITDIYGHVLEAGNITLQSGVPINTQQQFPSMGTSGVINQADK

SEQ ID NO: 31 - protein, 351 aa, Acidovorax avenae
MAMVAMRRTTLEGSMPKSLLSVFGRRCALAWGWLALAVALPVAAQGTPGETYTGRGTFYGYDGGGNCSLPFPEHVLTVAINDSDYQGSQACGAYLEVLNPATSKKVVVRVDNRCPDCPPHGLDLAIPALAQIAPIDAGIVSLRWRYVSGPDTPASVVFKEGSSASWSALQVRNQRNAVASLAYRASGSGGTYVPLERQMYNYFLAPGGMGPGPFDLKITDVFGQVLEVSGVPLSVGPELSLGVQFPPVLPAAGSAWSVQDSEPPAPATGPVTYATSFNSDWGQGYCMNVTVTNPHAGPVDWAVRIPVSGTVYNAWDSQVTQVGNELSVQGAAWNRTLQPGASAQFGFCANR

SEQ ID NO: 32 - protein, 727 aa, Clavibacter michiganensis
MTVRQVSVPLVSKLFLFFALAVGATFGAFAAPALAATAAGTGAVASPPGWLHTAGGKIVTASGAPYTIRGIAWFGMETSSCAPHGLDTITLAGGMQHIKQLGFTTVRLPFSNQCLAASGVTGVDADPSLAGLTPLQVMDHVVASAKAAGLDVILDQHRPDSGGQSELWYTSEYPESRWISDWRMLAKRYASDPTVIGVDLHNEPHGAATWGTGAATTDWRAAAERGGNAVLAENPKLLVLVEGIDHQADGTGTWWGGALDSAATASVRLTVANRVVYSPHDYPSTIYGQPWFSASNYPTNLPGIWDAHWGYLAKKDIAPVLVGEFGTKLETASDKQWLNTLVGYLSSTGISSSFWAFNPDSGDTGGIVKSDWVTPEQAKLDALAPILHPAPGAGPGSSGSGSQPGPAKPGAVSVKWQPGGSWASGYVANLDVTATGAVTGWTVSWASPKTTSVVNSWGMRCSVASNTVTCTSTDWASKLAAGQTVRVGVQLAGGPAPASPRISVTAAGTPPSQATPPSQATPPSQATTHGRATHYSLGTGNTIANGNCSMPAVPADRMYVAVSSPEYGGAAACGSHLLVTGPKGTVRVQIVDQCHECEIGHLDLSEEAFRAIGDFDAGIIPISYTTVRDPSVPAVAVRVKEGSSRWWAGLQILNAGNRIDRVEVRAGGQWLPLSRTDYGYWVTPSPIPDGPMTVRVTDQYGRSVVLPGIRIAPGETQSTARRIYQMH

SEQ ID NO: 33 - protein, 746 aa, Clavibacter michiganensis
MRKASVEVVFSAAGWLPYDPYMTFRQVRASLVLRLVLLLALVVGTTSAAFAAPVSAATVAGPVAAASSPGWLHTAGGKIVTASGAPYTIRGIAWFGMETSSCAPHGLDTITLAGGMQHIKQMGFTTVRLPFSNQCLAASGVTGVSADPSLAGLTPLQVMDHVVASAKSAGLDVILDQHRPDSGGQSELWYTSQYPESRWISDWRMLAKRYAAEPTVIGVDLHNEPHGAATWGTGAATTDWRAAAERGGNAVLAENPNLLVLVEGIDHEADGSGTWWGGALGLVGNAPVRLSVANRVVYSPHDYPSTIYGQSWFSASNYPANLPGIWDAHWGYLAKKDIAPVLVGEFGTKFETTSDKQWLNTLVGYLSSTGISSSFWAFNPNSGDTGGIVKSDWVTPEQAKLDALAPILHPSPGSGPGSGGSGSQPAPQPDPANPGAVSAKWQPGSSWASGYVANIDVTATAAVTGWTVSWASPGTTRVVNSWGMRCSVASGTVSCTGTDWASKLAAGQTVHVGLQASGGPAPSSPRLTATAAAVPPAQPTPPARPTTHGRATHYSLGQGNTIANGNCSMPAVPADRMYVAVSSPEYSGAAACGTFLDVTGPKGTVRVQVADQCHGCEVGHLDLSEEAFRALGDFNAGIIPISYVTVRDPAGPTVAIRVKEGSSRWWAGLQVLNAGNRIDRVEIQAGRQWLPLTRTDYGYWVTPSPIQDGPLTVKVTDQYGRAVVLPGLRMAPGEIQRTASRFYPVH

SEQ ID NO: 34 - protein, 253 aa, Clavibacter michiganensis
MTRASALPGRPRAPRRPFTAARRRILSAALAVLVAVAGPAMAASAASAAPAAGPARVSGYATHYSLGPDGRTTNGNCSLPAIPKDRLYVAVGPDLYAGGAGCGSYFDVTGPHGTVRVEVADSCHECVHGHLDLSEEAFRAIGDYDAGIITTSYVPVAASTVPPLSFRFKDGSSAYWAALQVLDAGVRLRSVELWVGARWVPLSLTDYGYWLAPGYVGAGPFTVRVTDTTGRTATVQGIVLDPMRLQHTASRLR

SEQ ID NO: 35 - protein, 232 aa, Bacillus subtilis
MKKIMSAFVGMVLLTIFCFSPQASAAYDDLHEGYATYTGSGYSGGAFLLDPIPSDMEITAINPADLNYGGVKAALAGSYLEVEGPKGKTTVYVTDLYPEGARGALDLSPNAFRKIGNMKDGKINIKWRVVKAPITGNFTYRIKEGSSRWWAAIQVRNHKYPVMKMEYEKDGKWINMEKMDYNHFVSTNLGTGSLKVRMTDIRGKVVKDTIPKLPESGTSKAYTVPGHVQFPE

SEQ ID NO: 36 - protein, 220 aa, Bacillus pumilus
MLSFAVPNMKAASVLDEVRESYATYTGSGYSGGAALLDPIPSDMKITALNPHDYNYKGINAALAGAYLEVKGPKGKTVVYVTDKYPEGAKGALDLSHNAFAKIGNMADGKIPISWKIVKAPISGNVVYRIKEGSSKWWAAIQVRNHKYPIMKMEYYKDGQWVNMEKTDYNHFLGFNMGSKSLPVRITDIRGVIVKDKLPALPSTAASQAYTVKGNVQLPN

SEQ ID NO: 37 - protein, 666 aa, Xanthomonas campestris
MTSEQMPSLAERALHGTGSVAPRQAAQRPLRAADGQPGLWHAGTASRALPGSCALLSPPAQAASHATVHNTLQKTSMPSHIHFLIALATLIPVTAPAMQVSTQAPLVDATGQTLHIRGVTWPGFDRAGLAAVGMRNNTLAQLLDRMQASDINAVRVPVCAAVLQRAPVAAAEVAGDSTLRGLDSLQLLDAVVHAASQRGMQVMFAFADGGCDDRAPLLGAQQQAWTRGLVTLARRYGGNANVLGIDLGSSGYRNASWAGNAADQDWNRVASRAVARVLAQAPRWVVGVEGVGSNAVCSDPERKAPGSNLQPFACVPLDIARRHLVLMPKLAGPDRDTTDAFAAPGFAQALPAMWQRDFGQFAIDHAVVPVSLGGGLGDGDPRDPAWQTALSGYLANAGIRSAFLGSWETGNANNGGLLAPDGSPRADKLLILRHAWGRLPVMPAIATATGDSTKNASGKKPWNSTFTGTATVTGSGYSGGALLLDPIPSDAFITALNPVQLNFGGVKAALAGAYLQVNGPKGTTTVYVTDLYPEGASGGLDLSHNAFAAIGDMVQGRIPISWKVVRAPVTGNLQYRIKEGSSRWWAAIQVRNHAYPVVKLEVKQGSTWKNLQKMDYNHFLGEQLGNQPLTLRITDIRGKVLTDTLPRLPEDGSKPAYFEPGHVQFP

SEQ ID NO: 38 - protein, 590 aa, Xanthomonas oryzae
MLSHIHFLIALATLVPVTAPAMQVSTQAPLVDATGQTLHIRGVTWPGFDRAGLTAVGMRNNTLAQLLDRMQASDINAVRVPVCAAVLQRAPVAAAEVAGDSTLRGLDSLQLLDAVVHAATQRGMHVMFAFADGGCDDRAPLLGAQQQAWTRGLVTLARRYGGNANVLGIDLGSSGYRNASWAGNAADQNWNRVASRAAAMVLAQAPRWVVGVEGVGSNAVCSDPGRKALGSNLQPFACVPLDIAPRHLVLMPKLAGPDRDTTDAFAAPGFAQALPAMWQRDFGQFAIDHTVVPVSLGGGLGDGDPRDPAWQTALSGYLANTGMRSAFLGSWETSNANNGGLLAPDGSPRADKLLILRHAWGMLPVMPAIATATGDSTEPAIGKNAWNSTFTGTATPTGSGYSGGAVLLDPIPSDAFITALNSTQMNFGGIKAALAGAYLEVTGPKGKTTVYVTDLYPDGASGGLDLSYNAFAAIGNLADGHIPISWKVVRAPITGNVQYRIKEGSSRYWAAIQVRHHIYPVVKLEVKQGSTWTSLPKTPYNHFVGTNLGNKPLSIRITDIRGKVIADKIPALPEYGGSAAYFEPGHVQFP

SEQ ID NO: 39 - protein, 274 aa, Ralstonia solanacearum
MSCFKKWQWIAIMLLMLPVLSARANGQTTAAGNVAAAQGSCLGTGTAAGNGATTPANAWDSTFTGIATATGSGYSGGAFLLDPIAKDAEITALNPVQANLGGIRAAMAGAYLRVQGPKGCTTVYVTDLYPEAASGGLDLSYNAFSKIGDLQQGRIPIRWKLIAAPVTGNVVYRIKEGSTMWWAAIQVRNHTYPVVKLEVFQGKAWVSLPKADYNHFVGTQLGDKPLVIRITDIRGRILVDKLPPLLKDCTPKSANQPSPCSKPYFVQGNVQFPE

SEQ ID NO: 40 - protein, 256 aa, Ralstonia solanacearum
MLLSLSVWSLHANGQVVAADNATAAQPSCLGTGTTAPANTWGSTFTGIATATGSGYSGGAFLLDPITKDREITALNPAQANLGGIPAAMAGAYLRVQGPKGCTTVYVTDLYPEAASGGLDLSYNAFAKIGDLQQGRIPVQWRLIPGPVTGNVVYRIKEGSTMWWAAIQVRNHTFPVVKLEVFQGKAWVSLPKADYNHFVGTQLGDKPLVIRITDIRGRILVDKLPPLLKDCTPQKAGEASPCSKPYFVQGKVQFSE

SEQ ID NO: 41 - protein, 584 aa, Xylella fastidiosa
MRRLAILFWLATSGCVASAMYGGTVDAQNAVSDTHFVEPLRGVNWRGLETAQHLPQGLDQRPWREVLDQMQSLGINAIRLPLCSDTLHGAMPTNLDLVRNPDLKGRTALQIADAIMDEAGKRGMRVLLAYHGVECPTDGNPLLRSVDESEHQWISDMQFITSHYRAQQKVVIGVDLADMAYHRPFQSGGDSTPDWNRVVERAAAAILAMNPDWLIGVQPVGLNPPCLDASAPISDDNIKSQHCVQLRIPARNLLLMPRFAGTDMDTEAALGAFSGKQTVLPNSLDATDAEQLAHRIDALLAFGIRQGFYGSWMTSAQMPFGLLDNDGRTPRTALIAQLHRWWGVSRVDVASENAATKNQTTTDTNGCVTGDSSVPLNGWDTSFSGVATYTYTGYKGGALMLDPIQSHVQITALNPTQLNLGGIPAAMAGAYLRVQGPKGSTTVYVTDLYPTGSSGGLDLSPNAFASIGNMAQGRIPVQWKVVSAPVSGNLIYRVKKGSSGWWAAIQVREHRYPVLKLEICQDGTWLNLPKRNYNYFVGTRLGNQPLSMRMTDIRGQTLIDTLPALPKKASSKAYSVNGNVQFSE

SEQ ID NO: 42 - protein, 229 aa, Pectobacterium atrosepticum
MNKITSLALSALCVIPLINTAHAQWELDDICYGYATATGSGYQGGALLLDPIPQNMEITALNRSQLDYRGVKASLAGAYLKVNGPKGSTVVYVTDLYPEGGDCALDLSFNAFEKIGDLRDGKINIDWTLIEAPVNGNVIYRIKEGSNPYWAAVQFRNVKYPVIEMKYMRDNQWVAAQKTDYNHFIVEHVGMNDIPIEFTDVKGNVLSDTLPPMSQSTSSAYLITGNVQL

SEQ ID NO: 43 - protein, 232 aa, Bacillus licheniformis
MKKKISILITAMFLTILCFSPQASAAYNSLHTGYATYTGSGYSGGALLLDPIPSNMKITALNPTDMNYRGVKAALAGAYLRVEGPKGKTTVYVTDLYPEGAPGALDLSPNAFREIGDMKDGKIDIKWRIVKAPITGNFTYRIKEGSSQWWAAIQVRNHKYPVMKMEYYKDGKWINMEKTDYNHFVSTNLGTSPLKVRITDIRGKVVKDTIKKLPENGTSSAYTVPGKVQFPD

SEQ ID NO: 44 - protein, 251 aa, Nakamurella multipartita
MATGVEPINPEFSLLTESTTPLAGARRPRRRTAVALLIGAVAATLLLAAPPAAAATSGKATHYEATGGSTTNGNCSFPALPADKLYVAVSPTEYAKGAACGTYLDVTGPRGTVRVVVMDKCPECATGHIDLSKTAFAKIGALSDGIIPVTYSTVKNPTVPALNFRFKNGSSRWWFALQVLNHGNRLQSVSVQVNGKWVAATLADYNYWIYQPGAGPGPYTLLVKDIYGQQAIVSGVTMSPTLVQTTTARLK

SEQ ID NO: 45 - protein, 303 aa, Micromonospora sp.
MKENADMPPLHPRRPWRIAFASAVSAVVLASVAGCGGPSPAWHAAPTPAAGTSTPAVGKSGPGFPTAAADATTPATTPTTARPSATGSPSPKSSGVAPLAGRIRPNVTYRGKATFYDAGDGGGACLFGPASDLMIGAMNQTDYESAKACGAYVLVKAANGNSVTVRITNLCPLPCAPGQIDLSPQAFAKLANRSLGEVPITWKLLSPSMSDTISIRYKVGSSQWWCGIQAIGHRNPVALLEVRTSSGWRQLPRPEYNYFISANGSGCGGPIRITDIYGEKLTISGIKLKPDVVQPTRVQFARH

SEQ ID NO: 46 - protein, 326 aa, Catenulispora acidiphila
MKATHPTTHRSRWLPISTAAGLLVVAGLIAGLAMGSSSRHTADPPPAATGFATTGSGMTATPNSAPTSAEPSAPAVASGTASSASSPTVAAAHKTPGKTSTTSQTSATPTHGGAPSPTATSLAGRVQPGVTYQGVATEYSAADGDGACLFGPAADMMIAAMNELDYQNSEACGAHVLVRAANGATITVLITNECPYPCAPGQLDLSQQAFAKLADPKAGRISVTWQLVSPAAAGNVSIRYKVGSSQYWCGIQVIGERNPVARLEVAAGSGWQQLPRASYNYFLSANGAGCGKAVRITDIFGQQVTTAALPVEPDVIQPAGVQFSRH

SEQ ID NO: 47 - protein, 231 aa, Dickeya zeae
MMKLKSVLLASSVLFLPLTQQAQAAWELGDICYGYATATGSGYSGGALLLDPIPSNMEITALNRAQLDYKGVKAALAGAYLKVTGPKGSTIVYVTDLYPEGGDCALDLSFNAFEKIGNLQDGKINIQWELVKAPVSGNVVYRVKEGSNPYWAAVQFRNVKYPIIEMKYLRGTQWVSAPKTDYNHFILEFVGKNDIPIEFTDIKGNILSDTLPPMSDSTSSAYLITGKVQLP

SEQ ID NO: 48 - protein, 267 aa, Oryza sativa
MASSSLLLACVVVAAMVSAVSCGPPKVPPGPNITTSYGDKWLEAKATWYGAPKGAGPKDNGGACGYKDVDKAPFLGMNSCGNDPIFKDGKGCGSCFEIKCSKPEACSDKPALIHVTDMNDEPIAAYHFDLSGLAFGAMAKDGKDEELRKAGIIDTQFRRVKCKYPADTKITFHIEKASNPNYLALLVKYVAGDGDVVEVEIKEKGSEEWKALKESWGAIWRIDTPKPLKGPFSVRVTTEGGEKIIAEDAIPDGWKADSVYKSNVQAK

SEQ ID NO: 49 - DNA, 39 bp, Artificial Sequence
  BsEXLX1N primer
gaaggagatataaggatggcatatgacgacctgcatgaa

SEQ ID NO: 50 - DNA, 36 bp, Artificial Sequence
  BsEXLX1C primer
atgatggtaatggtgttcaggaaactgaacatggcc

SEQ ID NO: 51 - DNA, 31 bp, Artificial Sequence
  StEXLX1n stEXLX1C primer
ctttttcaggctaaactgctcagcaccatcg

SEQ ID NO: 52 - DNA, 70 bp, Artificial Sequence
  StEXLX2N primer
atgtctgaacctgacgactccggctttactaacgatcctgaaggcgaggttcgtgctctgggtgaatttc

SEQ ID NO: 53 - DNA, 20 bp, Artificial Sequence
  XoEXLX1N primer
atgcaggtcagtacgcaagc

SEQ ID NO: 54 - DNA, 19 bp, Artificial Sequence
  XoEXLX1C primer
gggaaactgtacgtggccg

SEQ ID NO: 55 - DNA, 22 bp, Artificial Sequence
  HcEXLX1N primer
gaaaatcgagtttctgcgactc

SEQ ID NO: 56 - DNA, 24 bp, Artificial Sequence
  HcEXLX1C primer
tttgtctgcctgattaataacgcc

SEQ ID NO: 57 - protein, 242 aa, Zea mays
KVPPGPNITTNYNGKWLTARATWYGQPNGAGAPDNGGACGIKNVNLPPYSGMTACGNVPIFKDGKGCGSCYEVRCKEKPECSGNPVTVYITDMNYEPIAPYHFDLSGKAFGSLAKPGLNDKIRHCGIMDVEFRRVRCKYPAGQKIVFHIEKGCNPNYLAVLVKYVADDGDIVLMEIQDKLSAEWKPMKLSWGAIWRMDTAKALKGPFSIRLTSESGKKVIAKDVIPANWRPDAVYTSNVQFY

(19) United States
(12) Patent Application Publication
(10) Pub. No.: US 2010/0173386 A1
(43) Pub. Date: Jul. 8, 2010

(54) PROKARYOTIC EXPANSIN PROTEIN ACTIVATING CELLULOSE EXPANSION AND CELLULOSE - DEGRADING COMPOSITION COMPRISING THE SAME

(76) Inventors: Kyoung Heon Kim, Seoul (KR); In-Geol Choi, Seoul (KR); Hee Jin Lee, Seoul (KR); Eun Sil Kim, Seoul (KR)

Correspondence Address: CHRISTIE, PARKER & HALE, LLP, PO BOX 7068, PASADENA, CA, 91109-7068, US

(21) Appl. No.: 12/559,370

(22) Filed: Sep. 14, 2009

Related U.S. Application Data
(60) Provisional application No. 61/096,666, filed on Sep. 12, 2008.

(51) Int. Cl. D21C 9/00; C07K 14/00; C12N 9/42; C12N 9/24

(52) U.S. Cl. 435/209; 530/350; 435/200; 435/277

(57) ABSTRACT

Provided is a prokaryotic expansin protein capable of expanding cellulose. The prokaryotic expansin protein performs the same function as existing plant expansin proteins. The prokaryotic expansin protein can be produced at greatly reduced cost on an industrial scale, unlike plant expansin proteins. Particularly, when lignocellulosic biomass is hydrolyzed into glucose using the prokaryotic expansin protein and cellulase, the hydrolysis rate of cellulose can be markedly increased by the cellulase, resulting in improved yield of the glucose. In actuality, the use of the prokaryotic expansin enables the production of bioenergy at low cost with a reduced amount of enzyme used. The prokaryotic expansin protein softens the textures of pulp, cotton fibers (e.g., jeans), etc. Therefore, the prokaryotic expansin protein can be used for various purposes, such as biopulping and biostoning.

### CROSS-REFERENCE TO RELATED APPLICATION

This application claims priority to and the benefit of U.S. Provisional Application 61/096,666 filed on Sep. 12, 2008, the entire content of which is incorporated herein by reference.

### INCORPORATION BY REFERENCE

The material in the text file entitled “64337SequenceList.txt”, created Sep. 12, 2009, and being 157,000 bytes, is herein incorporated by reference in its entirety.

### BACKGROUND OF THE INVENTION

1. Field of the Invention

The present invention relates to a prokaryotic expansin protein for activating the expansion of cellulose and a cellulose-degrading composition comprising the same. More specifically, the present invention relates to a prokaryotic expansin protein having cellulose expansion activity and a cellulose-degrading composition comprising the prokaryotic expansin protein.

2. Description of Related Art

Although more than five decades have passed since the early efforts towards utilizing lignocellulosic biomass by hydrolyzing cellulose into sugars that can be fermented into ethanol (Kim et al. 2005; Nguyen et al. 1998; Sherrad and Kressman 1945; Torget et al. 1991), the bioconversion of cellulosic biomass into biofuels has remained a challenge. It is because cellulose is well protected from chemical and biochemical attacks by a complex of hemicellulose and lignin. Therefore, biomass pretreatment and cellulolytic enzyme production are necessary but very costly steps in cellulosic ethanol production (Gregg et al. 1998; Lynd et al. 2008; Wingren et al. 2003). The development of efficient cellulolytic enzymes to increase the rate and extent of cellulose hydrolysis are therefore critical from an economic perspective.

Due to the large quantities of cellulase required in biomass hydrolysis, the high cellulase enzyme cost is the primary hindrance in the cost-effective processing of lignocellulose. According to Novozymes, 40- to 100-fold more enzyme mass is required to produce an equivalent amount of ethanol from lignocellulose than from starch (Merino and Cherry 2007). Enzymes acting on cellulose must overcome several obstacles for successful catalysis. They must access the insoluble substrate, disrupt the packing of the highly-ordered polymer and direct a single polymer chain into the active site cleft of the enzyme. Thus, concerted attacks by multiple enzymes are critical in cellulose hydrolysis.

Although the synergistic effects of cellulolytic enzymes such as cellobiohydrolases (CBHs) and endoglucanases (EGs) have been relatively well studied (Irwin et al. 1993; Medve et al. 1994; Woodward et al. 1988), no clear findings have emerged on the relationship between cellulololytic and non-hydrolytic proteins. To facilitate plant cell wall growth and penetration into the plant cell wall, some non-hydrolytic proteins such as plant expansins (Cosgrove 2000a; Cosgrove 2000b; McQueen-Mason and Cosgrove 1994), fungal swollenin, and an expansin-like protein from nematodes (Qin et al. 2004) are known to be capable of loosening or disrupting the packing of the plant cell wall and polysaccharides (such as cellulose and hemicellulose). The “disruption” activity is often measured by an extensometer in terms of plant cell wall- or filter paper-weakening activity (McQueen-Mason and Cosgrove 1994; Saloheimo et al. 2002). This disruption activity of expansins and expansin-like proteins are thought to confer a synergistic effect on the enzymatic hydrolysis of cellulose by enabling the cellulose to be more accessible to enzyme. Such a synergistic effect of plant expansins with cellulase was first found in 2001 (Cosgrove 2001), but heterologous overexpression of intact plant expansins as active recombinant proteins has been unsuccessful. GR2 proteins, which are grass pollen group-2/3 allergens with sequence similarities of 25-40% with β-expansins, were recently reported to show a synergism with Trichoderma endoglucanase (Cosgrove 2007). Compared to expansins, GR2s have advantages that they are smaller (˜10 kDa) than expansins and easily expressed in active form in Escherichia coli. Recently, an unidentified non-hydrolytic protein purified from corn stover (55.78 kDa) was found to have a synergistic effect with cellulase on the hydrolysis of filter paper (Han and Chen 2007). However, expression of an active plant expansin in host organisms other than plants has proven unsuccessful, creating an obstacle in studying the application and characterization of expansins for the promotion of cellulose hydrolysis.

It is already known that plant expansin proteins assist in degrading cellulose due to their cellulose expansion activity. However, plant expansin proteins are not substantially expressed in host organisms, which makes it extremely difficult to produce them on an industrial scale (U.S. Pat. No. 6,326,470). The cell-wall loosening activity of expansin proteins originating from eukaryotes such as fungi is negligible, which makes it difficult to use them in industrial applications (U.S. Patent Publication No. 2003-104546).

### SUMMARY OF THE INVENTION

The present invention has been made in an effort to solve the problems of plant expansins as proteins expanding cellulose, and a first object of the present invention is to provide a prokaryotic expansin protein that has functional and structural similarities to plant expansins and can be produced on an industrial scale.

A second object of the present invention is to provide a cellulose-degrading composition comprising a prokaryotic expansin protein to improve the ability of a cellulose-degrading enzyme to degrade cellulose, and a method for degrading cellulose by using the cellulose-degrading composition.

A third object of the present invention is to provide use of the cellulose-degrading composition in various applications.

A fourth object of the present invention is to provide a method for producing the prokaryotic expansin protein on an industrial scale.

According to an aspect of the present invention, a prokaryotic expansin protein is provided having cellulose expansion activity.

In an embodiment, the prokaryote may be selected from the group consisting of Bacillus subtilis, Hahella chejuensis (KCTC 2396), Dictyostelium discoideum, Neosartorya fischeri, Aspergillus fumigatus, Aspergillus clavatus, Aspergillus oryzae, Aspergillus terreus, Penicillium chrysogenum, Aspergillus niger, Emericella nidulans, Magnaporthe grisea, Pyrenophora tritici-repentis, Phaeosphaeria nodorum, Sclerotinia sclerotiorum, Frankia sp., Streptomyces sviceus, Sorangium cellulosum, Stigmatella aurantiaca, Plesiocystis pacifica, Myxococcus xanthus, Leptothrix cholodnii, Roseiflexus sp., Roseiflexus castenholzii, Chloroflexus aurantiacus, Herpetosiphon aurantiacus, Acidovorax avenae subsp, Pectobacterium atrosepticum, Bacillus licheniformis, Xanthomonas campestris pv. campestris, Bacillus pumilus, Xanthomonas oryzae pv. oryzae, Ralstonia solanacearum, Clavibacter michiganensis, Xylella fastidiosa, Nakamurella multipartite, Micromonospora sp., Catenulispora acidiphila and Dickeya zeae. In a preferred embodiment, the prokaryote may be Bacillus subtilis, Stigmatella aurantiaca, Xanthomonas oryzae or Hahella chejuensis.

In an embodiment, the expansin protein may comprise an amino acid sequence selected from the group consisting of the amino acid sequences set forth in SEQ ID NOS: 1-47. All mutant proteins that have cellulose expansion activity through one or more mutations such as substitution, deletion, inversion and translocation to the proteins of SEQ ID NOS: 1-47 are also included in the scope of the present invention, so long as they do not impair the object of the present invention.

In an embodiment, the expansin protein may be the amino acid sequence set forth in SEQ ID NO: 20, 22, 30 or 35.

According to another aspect of the present invention, there is provided a cellulose-degrading composition comprising the prokaryotic expansin protein.

In an embodiment, the cellulose-degrading composition may further comprise a cellulose-degrading enzyme. In a preferred embodiment, the cellulose-degrading enzyme may be selected from the group consisting of cellulase, cellobiohydrolase, endoglucanase, cellobiase, and mixtures thereof.

In an embodiment, the cellulose-degrading composition may comprise 0.01 to 0.05 FPU of the cellulose-degrading enzyme and 200 to 400 μg of the prokaryotic expansin protein per g cellulose.

According to another aspect of the present invention, a method is provided for degrading cellulose, comprising reacting the cellulose-degrading composition with the cellulose at 40 to 60° C.

According to another aspect of the present invention, a method is provided for degrading cellulose, comprising reacting the cellulose-degrading composition with the cellulose at a pH not higher than 7.

According to another aspect of the present invention, a prokaryotic expansin protein is provided that has a homology of at least 60% to an amino acid sequence of an expansin (EXLX1) originating from Hahella chejuensis and has cellulose expansion activity.

According to another aspect of the present invention, a method is provided for producing bioenergy, comprising treating lignocellulosic biomass with the cellulose-degrading composition to degrade cellulose contained in the lignocellulosic biomass and eventually to produce reducing sugar.

According to another aspect of the present invention, a method is provided for softening paper or pulp, comprising treating the paper or pulp with the cellulose-degrading composition to degrade cellulose contained in the paper or pulp.

According to another aspect of the present invention, a method is provided for softening a fiber or fabric, comprising treating the fiber or fabric with the cellulose-degrading composition to degrade cellulose contained in the fiber or fabric.

According to yet another aspect of the present invention, a method is provided for producing a prokaryotic expansin protein on an industrial scale, the method comprising finding a prokaryotic protein having a structural similarity to a plant expansin, cloning the prokaryotic protein, and expressing the cloned prokaryotic protein in a strain.

### BRIEF DESCRIPTION OF THE DRAWINGS

FIG. 1. Comparison of structures of BsEXLX1 (PDB id: 2bh0) (SEQ ID NO: 35) and plant expansin from Zea mays, EXPB (Zea m 1) (PDB id: 2hcz) (SEQ ID NO: 57). Top: Stereogram of backbone superimposed backbone structures. BsEXLX1 and EXPB are shown in black and gray, respectively. Bottom: The sequence alignment of two proteins based on their structures. The identical amino acid residues in both proteins are indicated in dark gray. The secondary structure is shown with a cylinder (α-helix) or an arrow (β-strand) under the alignment based on the secondary structures annotated in EXPB structure (2hcz).

FIG. 2. SDS-PAGE of proteins from the recombinant E. coli. Lanes: M, protein markers with different masses; 1, insoluble proteins; 2, soluble proteins; 3, purified BsEXLX1 (24 kDa).

FIG. 3. Synergism of BsEXLX1 in cellulose hydrolysis when filter paper was incubated with 0.012 FPU of cellulase with or without 100 μg of BsEXLX1 or BSA per g of filter paper in a citrate buffer solution (pH 4.8) at 50° C. Experiments were performed in triplicate, and the data points and error bars indicate means±standard deviations.

FIG. 4. Effect of the amount of BsEXLX1 on synergism in cellulose hydrolysis. Filter paper was incubated with 0.06 FPU of cellulase with 0, 100, 200 or 300 μg of BsEXLX1 or BSA per g of filter paper in a citrate buffer solution (pH 4.8) at 50° C. Experiments were performed in triplicate, and the data points and error bars indicate means±standard deviations.

FIG. 5. Effect of the amount of StEXLX1 on synergism in cellulose hydrolysis. Filter paper was incubated with 0.06 FPU of cellulase with 0, 100, 200 or 300 μg of StEXLX1 per g of filter paper in a citrate buffer solution (pH 4.8) at 50° C. Experiments were performed in triplicate, and the data points and error bars indicate means±standard deviations.

FIG. 6. Effect of the amount of XoEXLX1 on synergism in cellulose hydrolysis. Filter paper was incubated with 0.06 FPU of cellulase with 0, 100, 200, 300 or 400 μg of XoEXLX1 per g of filter paper in a citrate buffer solution (pH 4.8) at 50° C. Experiments were performed in triplicate, and the data points and error bars indicate means±standard deviations.

FIG. 7. Effect of HcEXLX1 on synergism in cellulose hydrolysis. Filter paper was incubated with 0.06 FPU of cellulase with 0 and 360 μg of HcEXLX1 per g of filter paper in a citrate buffer solution (pH 4.8) at 50° C. Experiments were performed in triplicate, and the data points and error bars indicate means±standard deviations.

FIG. 8. Effect of cellulase loading on synergism in cellulose hydrolysis. Filter paper was incubated with 0.012, 0.06, 0.12 or 0.6 FPU of cellulase and with 0, 100, 200 or 300 μg of BsEXLX1 per g of filter paper in a citrate buffer solution (pH 4.8) at 50° C. for 48 h. Experiments were performed in triplicate, and the heights of bars and the error bars indicate means±standard deviations.

FIG. 9. SDS-PAGE of proteins from supernatants and filter papers incubated for 0, 6, 12, or 24 h with various mixtures of cellulase and/or BsEXLX1. The band strength at each incubation time was scanned, and the values for each protein were normalized by those at incubation time 0, where the band strengths of the major protein at 75 kDa were taken as the cellulase. (a) Supernatant from filter paper with cellulase alone. (b) Supernatant from filter paper with cellulase and BsEXLX1. (c) Supernatant from filter paper with BsEXLX1 alone. (d) Supernatant from filter paper with BSA alone. (e) Filter paper with cellulase alone. (f) Filter paper with cellulase and BsEXLX1. (g) Filter paper with BsEXLX1 alone. (h) Filter paper with BSA alone.

FIG. 10. Effect of reaction temperature on the synergism in cellulose hydrolysis. Filter paper was incubated with 0.06 FPU of cellulase and 300 μg BsEXLX1 per g of filter paper in a citrate buffer solution (pH 4.8) at 30, 40, 50, 60 or 70° C. for 36 h. Experiments were performed in triplicate, and the heights of bars and the error bars indicate means±standard deviations.

FIG. 11. Effect of pH of the reaction buffer on the synergism in cellulose hydrolysis. Filter paper was incubated with 0.06 FPU of cellulase with 300 μg BsEXLX1 per g of filter paper in a citrate buffer solution at pH 3, 4, 4.8, 6 or 7 at 50° C. for 36 h. Experiments were performed in triplicate, and the heights of bars and the error bars indicate means±standard deviations.

FIG. 12. Tensile strength of Whatman filter paper No. 3 incubated in buffer only (50 mM sodium acetate, pH 4.8), in buffers containing BsEXLX1 or BSA (both 600 μg/mL), or in 8 M urea. Experiments were performed in triplicate, and the heights of bars and the error bars indicate means±standard deviations.

FIG. 13. Scanning electron micrographs [(a)-(d): ×300, (e)-(i): ×900] of Whatman filter paper No. 3 incubated in different solutions at 30° C. for 1 h: (a) and (e), filter paper incubated with a buffer solution (50 mM sodium acetate, pH 4.8) containing BsEXLX1 (600 μg/mL); (b) and (f), filter paper incubated with 8 M urea solution; (c) and (h), filter paper incubated with a buffer solution (50 mM sodium acetate, pH 4.8) containing BSA (600 μg/mL); (d) and (i), filter paper incubated with a buffer solution (50 mM sodium acetate, pH 4.8) alone.

FIG. 14. Tree of molecular diversity using multiple sequence alignment.

FIG. 15A. A table containing some of the bacterial expansins together with their gene identification numbers; FIG. 15B. Tree of molecular diversity using multiple sequence alignment.

FIG. 16. Partial Amino acid sequence of StEXLX1 (SEQ ID NO: 22) originating from Stigmatella aurantiaca.

FIG. 17. Amino acid sequence of XoEXLX1 (SEQ ID NO: 38) originating from Xanthomonas oryzae.

FIG. 18. Amino acid sequence of HcEXLX1 (SEQ ID NO: 30) originating from Hahella chejuensis.

### DETAILED DESCRIPTION OF THE PREFERRED EMBODIMENTS

Existing plant expansins are very effective in improving the ability of cellulose-degrading enzymes to degrade cellulose due to their cellulose expansion activity. However, since plant expansins are not substantially expressed in host organisms, it is impossible to produce them on an industrial scale. Some eukaryotic proteins have been found to have structural and functional similarities to plant expansins, but their cellulose expansion activity is very low despite the possibility of mass production.

Thus, the present invention provides a prokaryotic expansin protein having a structural similarity to a plant expansin capable of expanding cellulose to achieve high degradation efficiency against cellulose substrate. The prokaryotic expansin protein of the present invention has similarities to a plant expansin in terms of its amino acid sequence, structure and function, and particularly enhances the degradation efficiency of a cellulose-degrading enzyme (particularly, cellulase) against cellulose substrate.

Plants have a rigid cell wall and need to loose their cell walls to add newly synthesized components. ‘Expansin’ is originally identified as a plant protein known to expand cell wall to facilitate synthesis of cell wall. Though expansin has no cell wall degrading activity, it binds to insoluble cell wall components (such as cellulose) giving loosening activity. The mechanism of expansin is not fully elucidated but seems breaking hydrogen bonds among insoluble cellulosic polysaccharides. Few patents are known to use a cell-wall loosening activity of plant expansins to various applications.

The expansin superfamily is highly conserved in plants and found in all plant genomes sequenced so far. In other eukaryotes such as fungi and worm, sequential and functional homologs are known. For instance, a swollenin protein having an expansin-like domain was identified from T. reesei based on its sequence similarity to plant expansin proteins. However, no prokaryotic protein similar to plant expansins was found yet.

Recently, atomic structure of a plant expansin (Zea mays EXPB, PDB code: 2hcz) (SEQ ID NO: 57) was determined. The structure of yoaJ protein of B. subtilis (YoaJ, PDB code: 1hb0) (SEQ ID NO: 35) also deposited in PDB as an expansin homolog showing high structural similarity but its function has not yet been verified. We used these structures as a template to search the sequence database and identified several bacterial proteins having sequence similarity to plant expansins. Most of them are annotated as proteins having unknown functions. SEQ ID NO: 48 is the protein sequence for the plant expansin ExpB1 from Oryza sativa japonica.

We identified these bacterial proteins and cloned several of them to test whether they have expansin functions. After overexpression in E. coli and purification of proteins, we verified the functions of bacterial proteins having a binding and loosening activity to plant cell wall components (e.g., insoluble cellulose). We tested these activities in terms of enhanced degradation of cellulosic materials by cellulases. We intend to use these prokaryotic proteins having sequential or functional similarity to plant expansin for the biomass pretreatments.

Structure-based sequence analysis—The remote homology relationship often can be obtained from a sequence model using 3D-structural information. We identified that the expansin structures contained ‘Barwin-like endoglucase’ (model #) and ‘PHL pollen allergen’ (model#) domains. We also found the corresponding domains (DPBB-PF03330 and Pollen allergen-PF01357) in Pfam, which is one of the comprehensive domain databases. Therefore, we regarded a protein as an expansin homolog if a protein has two sequence models in an open reading frame at the same time.

In addition, we searched public databases (e.g., Genbank) using yoaJ sequence of B. subtilis by Blast. We used the statistical significance (P-value) threshold for selecting homologs as 0.001 (P<0.001).

We identified for bacterial proteins functionally, sequentially or structurally homologous to the expansin superfamily, which have not reported yet. We began with seeking for bacterial expansins having a structural similarity to the plant β-expansin of Zea m 1 (also known as EXPB) deposited in the Protein Data Bank (PDB) (PDB id: 2hcz) (SEQ ID NO: 57). We found YoaJ protein from Bacillus subtilis, the structure of which was first determined and deposited by the group of Charlier in 2006 (Petrella et al. 2006). The YoaJ was named as BsEXLX1 by following the nomenclature of expansin (Kende et al. 2004), and the structure of BsEXLX1 was released in public but not published yet (PDB id: 2bh0) (SEQ ID NO: 35).

We sought for bacterial expansins having a structural similarity to the plant β-expansin of Zea m 1 (also known as EXPB) deposited in the Protein Data Bank (PDB) (PDB id: 2hcz). We found YoaJ protein from Bacillus subtilis, the structure of which was first determined and deposited by the group of Charlier in 2006 (Petrella et al. 2006). The YoaJ was named as BsEXLX1 by following the nomenclature of expansin (Kende et al. 2004), and the structure of BsEXLX1 was released in public but not published yet (PDB id: 2bh0).

In order to verify the predicted function of bacterial expansin homologs, which have not been done yet, we cloned and expressed bacterial proteins including BsEXLX1 in E. coli. The purified proteins were examined for its expansin-like functions in vitro and its roles in cellulose hydrolysis.

We found functional homologs of the expansin superfamily from other bacteria such as Stigmatella aurantiaca, Xanthomonas oryzae and Hahella chejuensis based on their homology to BsEXLX1. These expansin homologs were designated as StEXLX1, XoEXLX1 and HcEXLX1, respectively, and were examined for their synergistic activity with cellulase in cellulose hydrolysis.

Then, we searched against Uniprot DB (curated protein database) using the BsEXPX1 (Bacillus expansin homolog) to acquire 66 protein sequences (cutoff threshold E-value<0.001).

We used the cd-hit program to remove redundant sequences with a sequence identity as high as 90% (leaving the representative sequences only) from the 66 protein sequences to obtain a total of 55 sequences.

We removed the sequences derived from the same species from the 55 sequences to obtain a total of 47 sequences.

The criterion for the cutoff adopted in the present invention is based on probability. Accordingly, the cutoff is not absolutely defined. A cutoff of 0.001 is generally considered significant (this value implies that one of 1,000 sequences obtained through database search is not a real homolog). Details thereof can be found in http://www.jcsg.org/psat/help/document.html as a reference website. At the top of the website, the following is described: “Homologous protein sequences were obtained at E-value cutoff=0.001.”

By the above procedure, we found prokaryotic expansin proteins comprising the amino acid sequences set forth in SEQ ID NOS: 1-47. The prokaryotic expansin proteins had cellulose expansion activity at levels similar to those of plant expansin proteins, as confirmed through a series of experiments, which will be described below. A prokaryotic expansin protein that has a homology of at least 60% to an amino acid sequence (which is set forth in SEQ ID NO: 30) of an expansin (EXLX1) originating from Hahella chejuensis and has cellulose expansion activity may be included in the scope of the present invention. Further, all mutant proteins that have cellulose expansion activity through one or more mutations such as substitution, deletion, inversion and translocation to the proteins of SEQ ID NOS: 1-47 are also included in the scope of the present invention, so long as they do not impair the object of the present invention.

The prokaryote may be selected from the group consisting of, but not limited to, Bacillus subtilis (SEQ ID NO: 35), Hahella chejuensis (KCTC 2396, SEQ ID NO: 30), Dictyostelium discoideum, se (SEQ ID NOS: 1, 2, 3), Neosartorya fischeri (SEQ ID NO: 4), Aspergillus fumigatus (SEQ ID NO: 5), Aspergillus clavatus (SEQ ID NO: 6), Aspergillus oryzae (SEQ ID NO: 7), Aspergillus terreus (SEQ ID NO: 8), Penicillium chrysogenum (SEQ ID NO: 9), Aspergillus niger (SEQ ID NO: 10), Emericella nidulans (SEQ ID NO: 11), Magnaporthe grisea (SEQ ID NO: 12), Pyrenophora tritici-repentis (SEQ ID NO: 13), Phaeosphaeria nodorum (SEQ ID NO: 14), Sclerotinia sclerotiorum (SEQ ID NO: 15), Frankia sp, (SEQ ID NO: 16), Streptomyces sviceus (SEQ ID NO: 17), Sorangium cellulosum (SEQ ID NOS: 18, 19), Stigmatella aurantiaca (SEQ ID NOS: 20, 22), Plesiocystis pacifica (SEQ ID NO: 21), Myxococcus xanthus (SEQ ID NO: 23), Leptothrix cholodnii (SEQ ID NO: 24), Roseiflexus sp, (SEQ ID NO: 25), Roseiflexus castenholzii (SEQ ID NO: 26), Chloroflexus aurantiacus (SEQ ID NO: 27), Herpetosiphon aurantiacus (SEQ ID NOS: 28, 29), Acidovorax avenae subsp (SEQ ID NO: 31), Pectobacterium atrosepticum (SEQ ID NO: 42), Bacillus licheniformis (SEQ ID NO: 43), Xanthomonas campestris (SEQ ID NO: 37), Bacillus pumilus (SEQ ID NO: 36), Xanthomonas (SEQ ID NO: 38), Ralstonia solanacearum (SEQ ID NOS: 39, 40), Clavibacter michiganensis (SEQ ID NOS: 32, 33, 34), Xylella fastidiosa (SEQ ID NO: 41), Nakamurella multipartite (SEQ ID NO: 44), Micromonospora sp., (SEQ ID NO: 45), Catenulispora acidiphila (SEQ ID NO: 46) and Dickeya zeae (SEQ ID NO: 47). Any prokaryotic expansin protein capable of expanding cellulose while possessing a structural similarity to a plant expansin may be used in the present invention.

The prokaryotic expansin protein of the present invention may be isolated or purified. This isolation or purification can be accomplished by a suitable separation technique known in the art, for example, ion-exchange chromatography, affinity chromatography, hydrophobic separation, dialysis, protease treatment, ammonium sulfate precipitation, size-exclusion chromatography, filtration, gel electrophoresis or gradient separation, to remove whole cells, cell debris, extraneous proteins or unwanted proteins in the final composition.

The present invention also provides a cellulose-degrading composition comprising the prokaryotic expansin protein. The cellulose-degrading composition may further comprise a cellulose-degrading enzyme. Examples of cellulose-degrading enzymes suitable for use in the cellulose-degrading composition include cellulase, cellobiohydrolase, endoglucanase and cellobiase. However, the use of the expensive cellulose-degrading enzymes inevitably increases the price of final products. Particularly, cellulase as the cellulose-degrading enzyme is used for the degradation of cellulose, a constituent polysaccharide of lignocellulosic biomass, into glucose, a monosaccharide. However, cellulase is too expensive for bioethanol, resulting in a steep rise in the price of the final product. Plant expansin proteins expand cellulose to make cellulase accessible to the cellulose, eventually reducing the amount of the cellulase used. As described above, the mass production and commercialization of plant expansin proteins are practically impossible. In contrast, the prokaryotic expansin protein of the present invention is expressed in a host organism while possessing substantially similar functions to plant expansin proteins, thus being suitable for mass production.

Preferably, the cellulose degradation composition of the present invention may comprise 0.01 to 0.05 FPU of the cellulose-degrading enzyme and 200 to 400 μg of the prokaryotic expansin protein per g cellulose (see Table 1).

The present invention also provides a method for degrading cellulose. Specifically, the method comprises reacting the cellulose-degrading composition with cellulose at 40-60° C. and pH≦7 for ≧48 hr.

The cellulose degradation composition of the present invention may further comprise one or more additives to improve the desired activity. Examples of such additives include activators, inhibitors, desirable ions, pH-adjusting compounds and other enzymes.

The present invention also provides a method for producing bioenergy. Specifically, the method comprises treating lignocellulosic biomass with the cellulose-degrading composition to degrade cellulose contained in the lignocellulosic biomass and eventually to produce reducing sugar.

The present invention also provides a method for softening paper or pulp. Specifically, the method comprises treating the paper or pulp with the cellulose-degrading composition to degrade cellulose contained in the paper or pulp.

The present invention also provides a method for softening a fiber or fabric. Specifically, the method comprises treating the fiber or fabric with the cellulose-degrading composition to degrade cellulose contained in the fiber or fabric.

The prokaryotic expansin protein used in the cellulose-degrading composition of the present invention has a structural similarity to an expansin, which is a cellulose cell wall loosening protein. The use of the prokaryotic expansin protein can increase the degradation efficiency of the cellulose-degrading enzyme against cellulose substrate. The prokaryotic protein has similarities to a plant expansin in terms of its amino acid sequence, structure and function, enhances the degradation efficiency of the cellulose-degrading enzyme (particularly, cellulase) against cellulose substrate, and can be produced on an industrial scale.

### EXAMPLE 1

### Bioinformatics Tools

A structural homolog search was performed against the PDB using the DaliLite program (Holm and Park 2000). To find expansin homologs in bacteria, we employed as a template the known structure of Zea m 1, which is a subset of β-expansins found in all groups of land plants (PDB id: 2hcz) (Yennawar et al. 2006). The alignment of the structure pair was done with DaliLite and UCSF chimera (Pettersen et al. 2004), and UCSF chimera was also used to draw the ribbon diagrams of protein structures.

### EXAMPLE 2-1

### Strains, Vectors, and Cloning

E. coli strain DH5α was used for plasmid cloning and amplification of the target gene, and E. coli strain BL21 (DE3) was used for expression of the cloned gene. The strains were grown at 37° C. with Luria-Bertani (LB) media (Difco, Sparks, Md., USA), and 50 μg/mL of ampicilin (Sigma, St. Louis, Mo., USA) was added for selecting transformants. The target gene was obtained by PCR amplification using the genomic DNA of B. subtilis as a template. The genomic DNA of B. subtilis was kindly provided by Berkeley Structural Genomics Center (BSGC, Berkeley, Calif., USA). The primer sequences for PCR reaction were BsEXLX1N (5′-GAAGGAGATATAAGGATGGCATATGACGACCTGCATGAA-3′, SEQ ID NO: 49) and BsEXLX1C (5′-ATGATGGTAATGGTGTTCAGGAAACTGAACATGGCC-3′, SEQ ID NO: 50), which were designed to remove a potential signal sequence in the amino-terminus of the target protein. The PCR primers were synthesized by IDT (Coralville, Iowa, USA). The PCR product was directly cloned into an expression vector by the ligation independent cloning (LIC) method (Aslanidis and Dejong 1990). We used a slightly modified expression vector from pET21a for LIC cloning, which was developed at BSGC (Graslund et al. 2008). The vector also had a 6-histidine tag at the carboxyl terminus for the affinity purification of expressed protein.

### Expression and Purification of Target Protein

E. coli BL21 (DE3) transformants containing recombinant plasmids were grown up to an absorbance of 1.0 at 600 nm in LB broth containing ampicillin. The cells were then induced with 1 mM of IPTG for additional 3 h to overexpress the recombinant protein. After the cells were collected by centrifugation at 5,000 rpm for 30 min, the cell pellet was resuspended in a lysis buffer (20 mM sodium phosphate and 0.5 M sodium chloride, pH 7.4) and disrupted by sonication. The suspension was centrifuged at 16,000 rpm for 20 min at 4° C. The supernatant was used as the soluble fraction of the protein, while the pellet was resuspended in a lysis buffer and used as the insoluble fraction. Both fractions were analyzed by 15% (w/w) SDS-PAGE followed by Coomassie staining to check their expression levels. The soluble fraction was loaded onto a His-trap column (GE Healthcare, Piscataway, N.J., USA) equilibrated with a lysis buffer. The column was washed with 10 column volumes of a buffer containing 25 mM imidazole, and the His-tagged target protein was eluted with a buffer containing 200 mM imidazole. The eluted protein was concentrated in an Amicon Ultra-15 Centrifugal Device (Millipore, Billerica, Mass., USA) and desalted with 20 mM sodium phosphate buffer. The protein was quantified using a protein assay kit (Pierce, USA). In the similar fashion, the bacterial expansin homologs from S. aurantiaca, X oryzae and H. chejuensis were cloned and expressed in E. coli BL21 (DE3).

### Overexpression of a Recombinant BsEXLX1

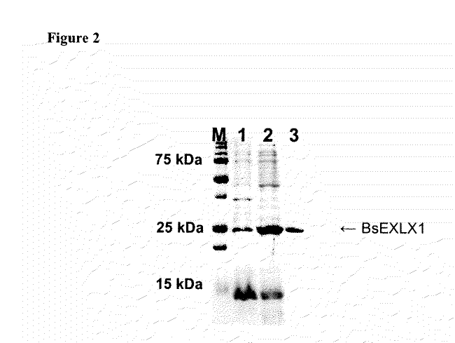

We cloned and overexpressed BsEXLX1 to see whether the structurally homologous protein had molecular functions similar to those of plant expansins, such as plant cell wall loosening, cellulose disruption (i.e., weakening) or cellulase activity enhancement. The potential signal sequence was removed from the amino terminus of BsEXLX1 to facilitate its heterologous expression in E. coli. The recombinant protein was expressed in E. coli and primarily existed in the soluble fraction (FIG. 2). The protein was purified with a 6-His tag located at the carboxyl-terminus of the protein. The typical yield of protein was about 10 mg protein from 1 L of culture broth.

Stigmatella aurantiaca, Xanthomonas oryzae and Hahella chejuensis were also used as sources of the superfamily of EXPB.

### EXAMPLE 2-2

### Strains, Vectors, and Cloning

E. coli strain DH5α was used for plasmid cloning and amplification of the target gene, and E. coli strain BL21 (DE3) was used for expression of the cloned gene. The strains were grown on LB media at 37° C. (Difco, Sparks, Md., USA), and 50 mg/mL of ampicilin (Sigma, St. Louis, Mo., USA) was added for selecting transformants. The target gene was obtained by PCR amplification using the genomic DNA of S. aurantica as a template. The genomic DNA of S. aurantica was kindly provided by Berkeley Structural Genomics Center (BSGC, Berkeley, Calif., USA). The primer sequences for PCR reaction were St EXLX1N and StEXLX1C (5′-CTTTTTCAGGCTAAACTGCTCAGCACCATCG-3′, SEQ ID NO: 51), which were designed to remove a potential expression inhibition sequence, in the amino-terminus of the target protein. The PCR primers were synthesized by IDT (Coralville, Iowa, USA). The PCR product was directly cloned into an expression vector by the ligation independent cloning (LIC) method (Aslanidis and Dejong 1990). We used a slightly modified expression vector from pET21a for LIC cloning, which was developed at BSGC (Graslund et al. 2008). The vector also had a 6-histidine tag at the carboxyl terminus for the affinity purification of expressed protein. StEXLX1 was expressed but not soluble type. So we had to redesign the N-term primer sequence to obtain the soluble format. The primer sequences for PCR reaction were StEXLX2N (5′-ATGTCTGAACCTGACGACTCCGGTTTACTAACGATCCTGAAGGCGAGGTTCGTGCTCTGGGTGAATTTC-3′, SEQ ID NO: 52) and StEXLX1C (as same as upper sequence) (SEQ ID NO: 51).

### Expression and Purification of Target Protein

E. coli BL21 (DE3) transformants containing recombinant plasmids were grown up to an absorbance of 1.0 at 600 nm in LB broth containing ampicillin. For soluble expression of proteins, we decreased the growth temperature to 16° C. after induction with final concentration of 1 mM IPTG and continued to grow for 16 h to overexpress the recombinant protein. After the cells were collected by centrifugation at 5,000 rpm for 30 min, the cell pellet was resuspended in a lysis buffer (20 mM sodium phosphate 300 mM sodium chloride, pH 7.4) and disrupted by sonication. The suspension was centrifuged at 16,000 rpm for 30 min at 4° C. The supernatant was used as the soluble fraction of the protein, while the pellet was resuspended in a lysis buffer and used as the insoluble fraction. Both fractions were analyzed by 15% (w/w) SDS-PAGE followed by Coomassie staining to check their expression levels. The soluble fraction was loaded onto a His-trap column (GE Healthcare, Piscataway, N.J., USA) equilibrated with a lysis buffer. The column was washed with 10 column volumes of a buffer containing 10 mM imidazole 20 mM sodium phosphate, and the His-tagged target protein was eluted with a buffer containing 200 mM imidazole. The eluted protein was concentrated in an Amicon Ultra-15 Centrifugal Device (Millipore, Billerica, Mass., USA) and desalted with 20 mM sodium phosphate buffer. The protein was quantified using a protein assay kit (Pierce, USA). The typical yield of protein was about 3 mg protein from 1 L of culture broth.

### EXAMPLE 2-3

### Strains, Vectors, and Cloning

E. coli strain DH5α was used for plasmid cloning and amplification of the target gene, and E. coli strain BL21 (DE3) was used for expression of the cloned gene. The strains were grown at 37° C. with LB media (Difco, Sparks, Md., USA), and 50 μg/mL of ampicilin (Sigma, St. Louis, Mo., USA) was added for selecting transformants. The target gene was obtained by PCR amplification using the genomic DNA of X. campestris as a template. The primer sequences for PCR reaction were XoEXLX1N (5′-ATGCAGGTCAGTACGCAAGC-3′, SEQ ID NO: 53) and XoEXLX1C (5′-GGGAAACTGTACGTGGCCG-3′, SEQ ID NO: 54), which were designed to remove a potential signal sequence in the amino-terminus of the target protein. The PCR primers were synthesized by IDT (Coralville, Iowa, USA). The PCR product was directly cloned into an expression vector by the ligation independent cloning (LIC) method (Aslanidis and Dejong 1990). We used a slightly modified expression vector from pET21a for LIC cloning, which was developed at BSGC (Graslund et al. 2008). The vector also had a 6-histidine tag at the carboxyl terminus for the affinity purification of expressed protein.

### Expression and Purification of Target Protein

E. coli BL21 (DE3) transformants containing recombinant plasmids were grown up to an absorbance of 1.0 at 600 nm in LB broth containing ampicillin. For soluble expression of proteins, we decreased the growth temperature to 16° C. after induction with final concentration of 1 mM IPTG and continued to grow for 12 h (relatively short time than others) to overexpress the recombinant protein without cutting by protease. After the cells were collected by centrifugation at 5,000 rpm for 30 min, the cell pellet was resuspended in a lysis buffer (20 mM sodium phosphate 300 mM sodium chloride, pH 7.0) and disrupted by sonication with 1 mM IPTG and protease inhibitor cocktail, (Roche, USA). The suspension was centrifuged at 16,000 rpm for 30 min at 4° C. The supernatant was used as the soluble fraction of the protein, while the pellet was resuspended in a lysis buffer and used as the insoluble fraction. Both fractions were analyzed by 15% (w/w) SDS-PAGE followed by Coomassie staining to check their expression levels. The soluble fraction was loaded onto a His-trap column (GE Healthcare, Piscataway, N.J., USA) equilibrated with a lysis buffer. The column was washed with 10 column volumes of a buffer containing 10 mM imidazole 20 mM sodium phosphate, and the His-tagged target protein was eluted with a buffer containing 200 mM imidazole. The eluted protein was concentrated in an Amicon Ultra-15 Centrifugal Device (Millipore, Billerica, Mass., USA) and desalted with 20 mM sodium phosphate buffer. The protein was quantified using a protein assay kit (Pierce, USA). The typical yield of protein was about 5 mg protein from 1 L of culture broth.

### EXAMPLEe 2-4

### Strains, Vectors, and Cloning

E. coli strain DH5α was used for plasmid cloning and amplification of the target gene, and E. coli strain BL21 (DE3) was used for expression of the cloned gene. The strains were grown at 37° C. with LB media (Difco, Sparks, Md., USA), and 50 μg/mL of ampicilin (Sigma, St. Louis, Mo., USA) was added for selecting transformants. The target gene was obtained by PCR amplification using the genomic DNA of H. Chejuensis as a template. The genomic DNA of H. Chejuensis was kindly provided by J. F. Kim at Korea Research Institute of Biosciences and Biotechnology (KRIBB). The primer sequences for PCR reaction were HcEXLX1N (5′-GAAAATCGAGTTTCTGCGACTC-3′, SEQ ID NO: 55) and HcEXLX1C (5′-TTTGTCTGCCTGATTAATAACGCC-3′, SEQ ID NO: 56), which were designed to remove a potential signal sequence in the amino-terminus of the target protein. The PCR primers were synthesized by IDT (Coralville, Iowa, USA). The PCR product was directly cloned into an expression vector by the ligation independent cloning (LIC) method (Aslanidis and Dejong 1990). We used a slightly modified expression vector from pET21a for LIC cloning, which was developed at BSGC (Graslund et al. 2008). The vector also had a 6-histidine tag at the carboxyl terminus for the affinity purification of expressed protein.

### Expression and Purification of Target Protein

E. coli BL21 (DE3) transformants containing recombinant plasmids were grown up to an absorbance of 1.0 at 600 nm in LB broth containing ampicillin (100 μg/L). For soluble expression of proteins, we decreased the growth temperature to 16° C. after induction with final concentration of 1 mM isopropyl thiogalactoside (IPTG) and continued to grow for 16 h to overexpress the recombinant protein. After the cells were collected by centrifugation at 5,000 rpm for 30 min, the cell pellet was resuspended in a lysis buffer (20 mM Tris-Cl and 300 mM sodium chloride, pH 8.0) and disrupted by sonication. The suspension was centrifuged at 16,000 rpm for 30 min at 4° C. The supernatant was used as the soluble fraction of the protein, while the pellet was resuspended in a lysis buffer and used as the insoluble fraction. Both fractions were analyzed by 15% (w/w) SDS-PAGE followed by Coomassie staining to check their expression levels. The soluble fraction was loaded onto a His-trap column (GE Healthcare, Piscataway, N.J., USA) equilibrated with a lysis buffer. The column was washed with 10 column volumes of a buffer containing 10 mM imidazole 20 mM Tris-Cl, and the His-tagged target protein was eluted with a buffer containing 200 mM imidazole. The eluted protein was concentrated in an Amicon Ultra-15 Centrifugal Device (Millipore, Billerica, Mass., USA) and desalted with 20 mM sodium phosphate buffer. The protein was quantified using a protein assay kit (Pierce, USA). The typical yield of protein was about 20 mg protein from 1 L of culture broth.

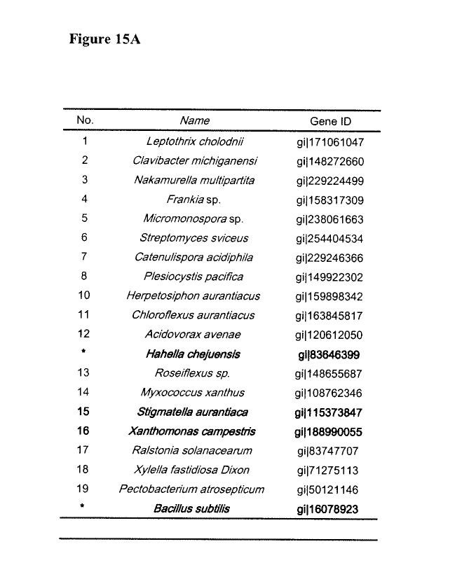

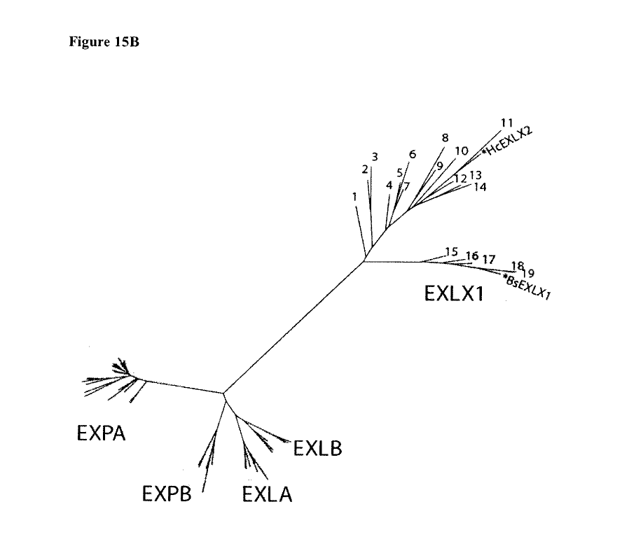

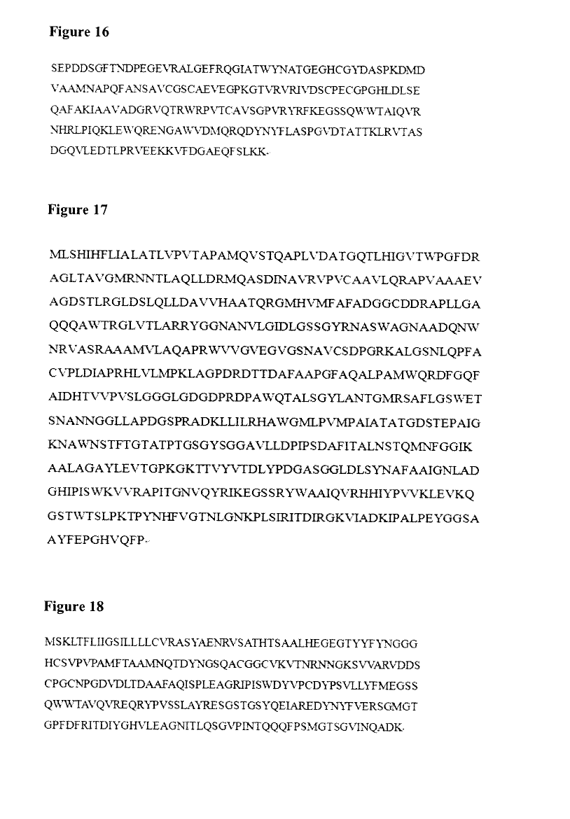

FIG. 15A shows a table containing some of the bacterial expansins together with their numbers and, FIG. 15B, an unroot tree illustrating the similarity and line between the bacterial expansins. As is known, plant expansins are antipodal to bacterial expansins in the unroot tree. The reason why the plant expansins are distinctly separated from the bacterial expansins in FIG. 16 is that BsEXLX1 is similar to StEXLX1. Unlike BsEXLX1 and StEXLX1, HcEXLX1 was derived from a marine microbe (Mara island, Cheju-do, Korea). It was found that HcEXLX1 belongs to a line different from the line of BsEXLX1 and StEXLX1. Specifically, XoEXLX1 has a size of 64 KDa and the other expansins have a size of 64 KDa. The reason for this size difference is that XoEXLX1 contains a cellulase domain. When the characteristics of the amino acid sequences shown in FIGS. 18 to 20 were compared using the ProtParam, the isoelectric points (pI) of StEXLX1 and HcEXLX1 were in the weakly acidic regions and the isoelectric points of XoEXLX1 and BsEXLX1 were in the alkaline regions. For this reason, the conditions necessary for the purification of the expansins are slightly different. First of all, the optimum conditions of the expansins should be substantially identical to those of enzymes to obtain synergistic effects with the enzymes. Generally, the alkaline expansins are stable in shape even in weakly acid conditions, whereas there is the risk that the weakly acidic expansins may precipitate. That is, it is more efficient to use an optimum expansin depending on the pH of a buffer using an enzyme. Further, it will be advantageous to use an expansin having an overlapping zone in the synergistic effect with an enzyme according to the characteristics (stability under harsh conditions) of a strain from which the expansin originates.

Plant expansins are broadly divided into α form, β form, Lα and Lβ by their lines. These forms are further subdivided (Sampedro, et al. 2006). Bacterial expansins are also divided into various forms by their lines. The lines of bacterial expansins began to be understood (It can be expected that a substrate and an enzyme on which BsEXLX1 creates a synergistic effect may be different from those on which HcEXLX1 creates a synergistic effect. Further, the optimal expansins may be varied depending on the type of biomass to be industrially used).

### EXAMPLE 3

### A Structural Homolog of a Maize Plant Expansin from Bacteria

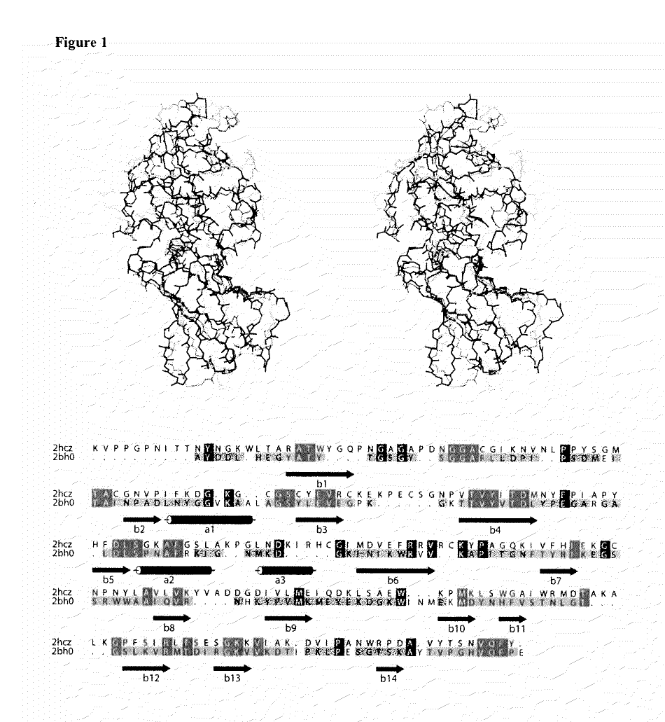

Although there are many known expansin domains in plant and fungi, no expansin-like domain has been identified in bacteria (Kende et al. 2004; Sampedro and Cosgrove 2005). The blast search against non-redundant protein database (nr) using the sequence of Zea m 1 EXPB did not give any significant bacterial protein hit on a criterion (p-value<0.001). Therefore, we rationalized that a structural homolog, which is difficult to be detected by a conventional sequence analysis because of sequential divergence (e.g., remote homology), may exist in bacteria. To verify this, we sought for a structural homolog of the recently solved structure of Zea m 1 EXPB (PDB id: 2hcz) against PDB (Yennawar et al. 2006). The information in PDB revealed that the structure of BsEXLX1 protein from B. subtilis (PDB id: 2bh0) was a plant expansin homolog. We used the structural database search program DaliLite to confirm that BsEXLX1 shows a high structural similarity to Zea m 1. The Z-score of the similarity was 20.7, which was statistically significant compared to the general criterion (Z≧2) for structural homolog selection; however, the sequence identity between two proteins was only 21% in structurally aligned regions. The root mean squared deviation (r.m.s.d.) between the structures of plant expansin Zea m 1 EXPB (2hcz) and BsEXLX1 (2bh0) was 2.4 Å, confirming that the two structures shared a nearly identical folding. The structure comparison and sequence alignment are shown in FIG. 1.

FIG. 1 shows diagrams comparing the structural similarity and amino acid sequences of β expansin from Zea mays (Zea m 1, PDB id: 2hcz) and BsEXLX1 (PDB id: 2bh0). The amino acid sequences of the β-expansin have a similarity of less than 20% to those of BsEXLX1 (bottom), but it can be seen from the stereogram of backbone superimposed backbone structures (top) that the structure of the beta-expansin is very similar to that of BsEXLX1. In conclusion, although the beta-expansin and BsEXLX1 have different sequences, they are expansin homologs having the same structural homology.

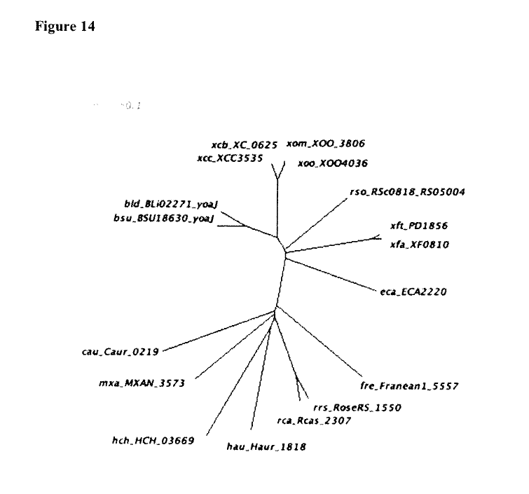

Appendices 1A, 1B, 2 and FIGS. 14 to 15A-B show or reference base sequence information. Appendix 1A shows a table of bacterial expansins and Appendix 1B shows similar specific domains between the bacterial expansins in Appendix 1A using multiple alignment (CLUSTALW) to find and compare domains of the bacterial expansins that are expected to perform the same functions as plant expansins.

In Appendix 2, graphic representations of a Fasta file obtained by analysis using CLUSTALW are expressed in JAVA. Domains corresponding to the similar amino acids are represented by different colors to clarify the structures of more similar domains.

FIGS. 14 and 15B show unroot trees as other methods for comparing the clarified structures.

The use of these methods is to distinguish the similarities and differences of the respective expansins, as already explained.

### EXAMPLE 4

### Enzymatic Hydrolysis of Cellulose

Cellulose hydrolysis was conducted on a microplate by a slight modification of a previously published rapid microassay (Berlin et al. 2006). Whatman No. 1 filter paper (Whatman, Florham Park, N.J., USA) cut into 6-mm diameter disks (2.5 mg each) was used as a substrate for cellulose hydrolysis. Each filter paper disk was placed in a 96-well PCR plate (Axygen, Union City, Calif., USA). The final concentrations of cellulase (Celluclast 1.5 L, Novozymes, Bagsvaerd, Denmark) and the bacterial expansin proteins in 0.05 M citrate buffer (pH 4.8) were 0-0.6 FPU per g cellulose and 0-300 μg per g cellulose, respectively, in 120 μL of the total reaction mixture. The 96-well plate was covered and incubated in a PCR machine (Peltier Thermal Cycler, Bio-Rad, Hercules, Calif., USA) at 50° C. for 48 h. Triplicate samples were taken every 12 h to assay for the reducing sugar levels with dinitrosalicylic acid (DNS) reagents (Adney and Baker 1996; Xiao et al. 2004) The absorbance of the color development at 540 nm by the reducing sugar, and DNS reagents was converted to reducing sugar concentration based on a glucose calibration curve. Triplicate data were expressed in means±standard deviations.

### Synergism of Cellulose Hydrolysis by BsEXLX1

Plant expansins were found to disrupt plant cell wall polysaccharides without having any hydrolytic activity, where they are thought to bind polysaccharides and break the hydrogen bonds between cellulose microfibrils (Cosgrove 2000a; Cosgrove 2000b; McQueen-Mason and Cosgrove 1994). The disruption effect on cellulose is often quantified as either the degree of extension or weakening of cellulose by an extensometer (McQueen-Mason and Cosgrove 1994; Yennawar et al. 2006). Heterologous expression of active expansins has not been reported in any organism other than plants so far. A nematode protein similar to the β-expansin of Arabidopsis thaliana has also been expressed in plants and has cellulose disruption activity against wheat coleoptiles and cucumber hypocotyls (Qin et al. 2004).

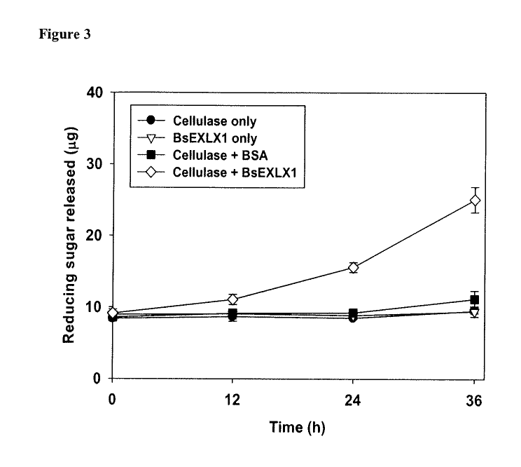

Although the structure of BsEXLX1 was determined and annotated as a structural homolog of Zea m1 EXPB by other group, its functional characterization as a bacterial expansin and application potential have not been studied yet. In this study, we conducted the molecular function of BsEXLX1 in vitro as a bacterial expansin. FIG. 3 shows the time course of enzymatic hydrolysis of filter paper by cellulase alone, BsEXLX1 alone or cellulase with BsEXLX1. Similarly to previous reports that have shown that plant expansins possess no hydrolytic activity (Cosgrove 2000a; Cosgrove 2000b; McQueen-Mason and Cosgrove 1994), incubation of filter paper with BsEXLX1 produced no significant amount of reducing sugar. When cellulase alone was added to the filter paper, the extent of sugar production with time was small due to the low cellulase loading (e.g., 0.012 FPU/g cellulose) in the reaction mixture. However, when BsEXLX1 (100 ug per g of cellulose) was added to the enzyme reaction mixture along with cellulase, the reducing sugar yield from the filter paper was 2.6-fold greater at 36 h of incubation than that of the control containing filter paper and cellulase alone. As a negative control, BSA was added with cellulase to filter paper to check whether any protein could affect the cellulose hydrolysis as BsEXLX1. However, the addition of BSA with cellulase did not result in significant change in sugar yield compared to the control without BSA.

### EXAMPLE 5

### Effect of Amount of BsEXLX1 on Synergism in Cellulose Hydrolysis

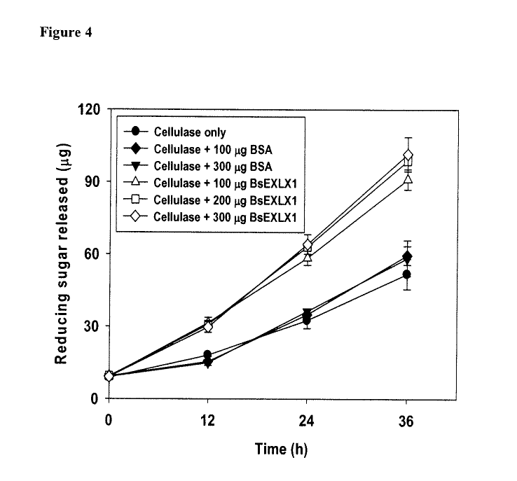

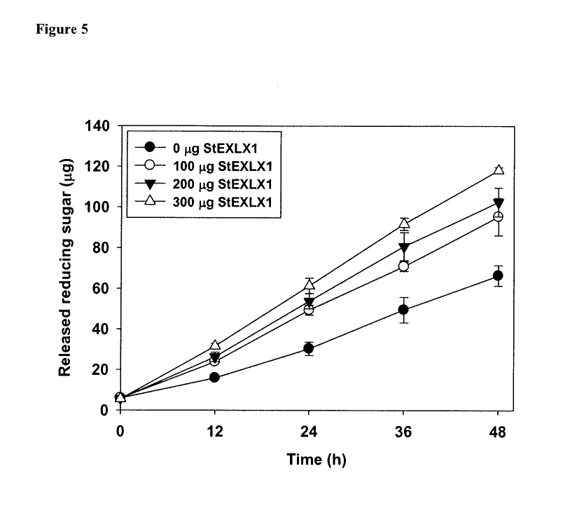

The relationship between the synergistic effect and the amount of BsEXLX1 is shown in FIG. 4. The reaction mixtures were composed of different concentrations of BsEXLX1 (0, 100, 200 or 300 μg) with 0.06 FPU of cellulase per g of filter paper. When 100 μg of BsEXLX1 per g of cellulose was added along with cellulase, the reducing sugar amount after 36 h was 1.7-fold greater than that without BsEXLX1. As the concentration of BsEXLX1 further increased, the synergistic activity also increased. However, a less significant increase in reducing sugar release was observed when the BsEXLX1 was elevated from 100 to 200 μg or from 200 to 300 μg, compared to elevation from 0 to 100 μg. Thus, the synergistic effect by BsEXLX1 was less obvious and appeared to be saturated by increasing the amount of BsEXLX1 added. As shown in FIGS. 5, 6 and 7, the synergistic effect of bacterial expansins derived from other bacteria, which are believed to belong to the superfamily of EXPB, were similar to that of EXLX1.

FIG. 5 shows the amounts of reducing sugar released after a mixture of StEXLX1, an expansin originating from Stigmatella aurantiaca, and 0.06 FPU of Novozymes Celluclast 1.5 L, a cellulase composite, per g of filter paper was applied to filter paper as cellulose substrate, followed by hydrolysis. As shown in FIG. 5, the amounts of reducing sugar released increased with increasing amount (100, 200 and 300 μg/g filter paper) of StEXLX when compared to a control containing no StEXLX1. These results demonstrate a synergistic effect of StEXLX1 and cellulase.

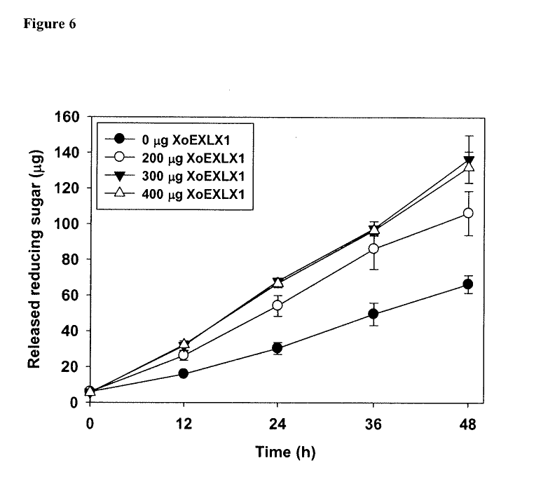

FIG. 6 shows the amounts of reducing sugar released after a mixture of XoEXLX1, an expansin originating from Xanthomonas campestris pv. campestris, and 0.06 FPU of Novozymes Celluclast 1.5 L, a cellulase composite, per g of filter paper was applied to filter paper as cellulose substrate, followed by hydrolysis. As shown in FIG. 6, the amounts of reducing sugar released increased with increasing amount (100, 200 and 300 μg/g filter paper) of XoEXLX1 when compared to a control containing no XoEXLX1. These results demonstrate a synergistic effect of XoEXLX1 and cellulase.

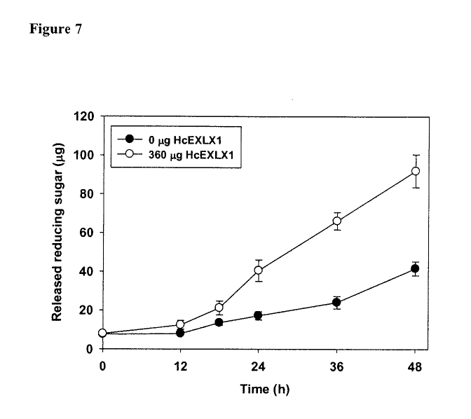

FIG. 7 shows the amount of reducing sugar released after a mixture of HcEXLX1, an expansin originating from Hahella chejuensis (KCTC 2396), and 0.06 FPU of Novozymes Celluclast 1.5 L, a cellulase composite, per g of filter paper was applied to filter paper as cellulose substrate, followed by hydrolysis. As shown in FIG. 7, the amount of reducing sugar released was larger when 360 μg of HcEXLX1 per g of filter paper was added than a control containing no HcEXLX1. These results demonstrate a synergistic effect of HcEXLX1 and cellulase.

### EXAMPLE 6

### Effect of Cellulase Loading on Synergism in Cellulose Hydrolysis

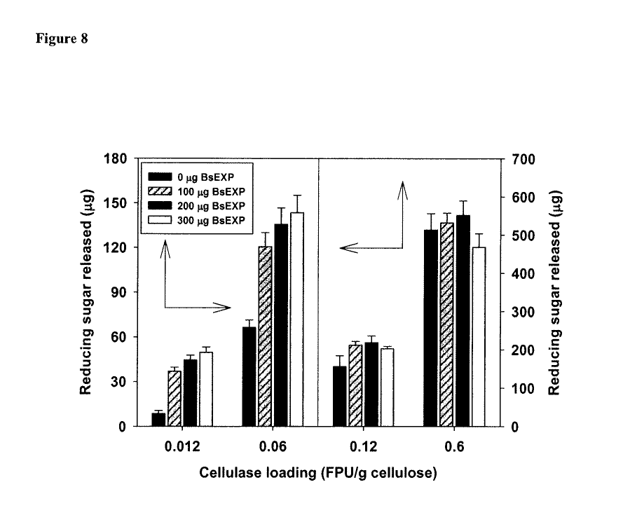

Cellulose hydrolysis by cellulase follows typical enzyme kinetics, in which the cellulase concentration increases with a fixed amount of substrate, the reaction rate also increases (Baker et al. 2000; Berlin et al. 2006). As shown in FIG. 8, the amount of reducing sugar released by cellulose hydrolysis significantly increased with an increase in enzyme loading in the tested range of 0.012-0.6 FPU per g filter paper. When the cellulase loading size was 0.012 or 0.06 FPU, we observed a noticeable difference in the reducing sugar accumulations between reaction mixtures containing filter paper and cellulase with or without BsEXLX1. When the amount of cellulase was further increased to 0.12 or 0.6 FPU per g filter paper, the synergistic effect became less significant. At these higher enzyme loadings, the co-incubation with BsEXLX1 at 300 μg per g filter paper gave a reducing sugar yield somewhat lower than the control without BsEXLX1.

The levels of cellulase loading in this study, 0.012-0.6 FPU per g cellulose, are lower than those generally used in testing enzyme digestibility of pretreated lignocellulose (which are usually higher than 5 FPU per g cellulose) (Rudolf et al. 2008; Yang et al. 2006). At 0.012 and 0.06 FPU of cellulase per filter paper, reducing sugar of up to 150 μg was produced from 2.5 mg of filter paper, where the sugar yield was equivalent to 5.5% of theoretical maximum. When the cellulase loading was 0.12-0.6 FPU, up to 600 μg of reducing sugar from 2.5 mg of filter paper, which is equivalent to 21.8% of theoretical maximum, was produced, but the synergism was insignificant. Due to the appearance of synergism at low cellulase loadings giving such low conversion ratios, at the present stage the synergistic effect of BsEXLX1 could not be industrially applicable to cellulose hydrolysis.

Such low sugar yields with low dosages of cellulase are common in the characterization or synergism study of cellulases with carbohydrate-binding modules (CBMs) or cellulose-binding domains (CBDs). For example, CBMs of Thermobifida fusca, E7 and E8 were added to the exoglucananse of the microorganism, Cel6B, the cellobiose yields from filter paper were only 5.0 and 5.1% of theoretical maximum after 168 h of hydrolysis at 50° C., respectively (Moser et al. 2008). In the study of synergism of Trichoderma reesei CBHs I and II, the conversion yield of Avicel ranged from 5 to 15% after 48 h of reaction (Medve et al. 1994). In a study on the role of CBMs contained in Cel5A of Bacillus sp., the reducing sugar yields from regenerated cellulose were up to 7.4 and 16.4% after 48 and 96 h of hydrolysis at 37° C. (Boraston et al. 2003). When using hybrids of CBDs and CelD of Clostridium thermocellum, the reducing sugar was produced up to 8.0 and 0.2% of theoretical maximum in the hydrolysis of Avicel and BMCC, respectively, for 48 h at 45° C. (Carrard et al. 2000).

Table 1 presents the summary of the synergistic activities of BsEXLX1 at a variety of combinations of cellulase and cellulose and at various reaction times.

TABLE 1
Synergistic activity (%) of cellulose hydrolysis by BsEXLX1 at
various ratios of BsEXLX1 and cellulase for a fixed amount
of filter paper

| | | ^{a}Synergistic | | |
|---|---|---|---|---|
| | Cellulase | activity (%) | | |
| BsEXLX1 | (FPU/g | Reaction time (h) | | |
| (μg/g cellulose) | cellulose) | 24 | 36 | 48 |
| 100 | 0.012 | 9.4 | 72.6 | 153.6 |
| | 0.06 | 55.0 | 60.0 | 66.6 |
| | 0.12 | 20.3 | 17.4 | 30.9 |
| | 0.6 | 6.8 | 5.9 | 2.5 |
| 200 | 0.012 | 37.3 | 129.0 | 205.4 |
| | 0.06 | 67.7 | 73.9 | 87.5 |
| | 0.12 | 27.7 | 22.0 | 34.7 |
| | 0.6 | 9.5 | 8.6 | 6.3 |
| 300 | 0.012 | 47.1 | 139.4 | 240.1 |
| | 0.06 | 70.7 | 78.9 | 98.3 |
| | 0.12 | 24.4 | 17.8 | 25.0 |
| | 0.6 | 2.8 | 6.4 | −9.8 |

^{a}Synergistic activity (%) = [{(reducing sugar released by BsEXLX1 and cellulase)/(reducing sugar released by cellulase alone + reducing sugar released by BsEXLX1 alone)} − 1] × 100

The ratio of a loading amount of BsEXLX1 and cellulase was found to be a critical determinant of the percent synergistic activity, which was calculated as the reducing sugar yield following co-incubation of BsEXLX1 and cellulase divided by the sum of the yields following individual incubation with cellulase alone or with BsEXLX1 alone. Of the various tested conditions shown in Table 1, the highest synergistic activity was 240%, which occurred at 0.012 FPU of cellulase and 300 μg of BsEXLX1 per g cellulose after 48 h of reaction time. This activity implies that the sugar yield from the addition of cellulase and BsEXLX1 together was 2.4-fold greater than the combined yields from BsEXLX1 alone and cellulase alone and 5.9-fold higher than the sugar yield from cellulase alone.

Synergism between different cellulases from the same microbial strains such as CBHs and EGs, have been well studied (Medve et al. 1994; Woodward et al. 1988), as has synergism in cellulases from different organisms (Irwin et al. 1993). However, the synergism by non-hydrolytic proteins has not been well characterized. When a plant β-expansin was combined with cellulase and applied to cellulose, this expansin caused an up to 13% increase in sugar conversion compared to that without expansin after 48 h (Baker et al. 2000).

A recombinant protein of GR2 from grass pollen was incubated with CBH I and EG II for filter paper for 18 h, an 8.3-fold sugar yield was obtained in comparison with incubating filter paper with enzymes only (Cosgrove 2007). However, the cellulase loading with GR2 was not specified in FPU but was presumed to be very low since the final sugar yields after 18 h were determined to be 0.6% and 4.8% of theoretical maximum. In the synergism study of cellulases and CBMs of T. fusca, the mixing of cellulases Cel6A and Cel6B with E7 showed only 12.9% and 7.5% increases of cellobiose yield, respectively, compared to the sugar yields with each cellulase alone after 168 h of reaction (Moser et al. 2008). In their study, the synergistic effect was observed at low sugar yields of 5.0% and 5.1% of theoretical maximum with Cel6B when combining with E7 and E8, respectively.

When an unknown non-hydrolytic protein purified from corn stover was co-incubated with 0.84 FPU of cellulase per cellulose, it showed 3.2-fold increase of glucose yield from filter paper compared to when incubated with cellulase only (Han and Chen 2007). As expected from the low cellulase loading in their experiments, the improvement of glucose yield (2.7 to 8.2% of theoretical maximum) took place at low levels of cellulose conversion.

### EXAMPLE 7

### Analysis of Cellulose Binding Activity of BsEXLX1

The incubation of proteins with filter paper for the cellulose binding assay was also performed in a PCR plate with 6-mm disk filter paper. One hundred microliters of each reaction mixture containing either 15 μg of bovine serum albumin (BSA; Sigma, St. Louis, Mo., USA) as a protein control, 10 μg of BsEXLX1 or 0.5 FPU of cellulase (equivalent to 54 μg of BSA) was incubated at 40° C. Samples (i.e., supernatants and filter paper disks) were taken at 0, 6, 12 and 24 h of incubation. The elutes from the filter paper, which were obtained by washing the paper twice with 20 μL of buffer, and the supernatants were analyzed by 15% SDS-PAGE followed by Coomassie staining.

As shown in Table 1, at a higher loading of cellulase such as 0.6 FPU per g cellulose, the increase of amount of BsEXLX1 to 300 μg rather decreased the synergistic effect or even resulted in the negative synergism. Such negative synergism was previously shown also in the interactions between CBH I and EG II or between EG I and EG II (Woodward et al. 1988). In both of these cases, EG was used at saturating levels, and the resulting negative synergism may have arisen from competition for binding sites in the substrate. Therefore, cellulase and BsEXLX1 may compete for binding sites of the filter paper at high concentrations of cellulase and BsEXLX1. Since expansins with cellulose disruption or plant cell well-loosening activities also possess binding activities against cellulose or other polysaccharides (Yennawar et al. 2006), the synergistic activity of BsEXLX1 may be initiated by its binding to the cellulose substrate first. If binding sites are shared by both cellulase and BsEXLX1, the competition between BsEXLX1 and cellulase for binding cellulose could exist. Therefore, the synergistic effect would become less distinctive at a higher loading of cellulase and BsEXLX1.

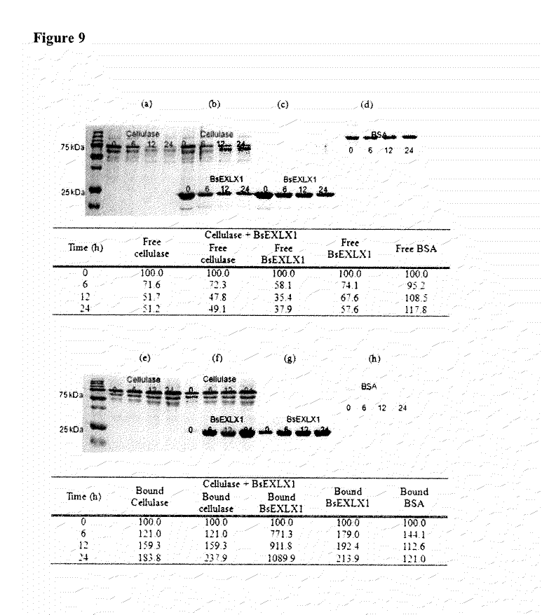

In the present study, we asked whether BsEXLX1 also had such binding activity for cellulose since a β-expansin from maize was shown to have binding activity against various polysaccharides (cellulose, xylan, galactan, and others) (Yennawar et al. 2006). As is shown in FIG. 9, BsEXLX1, cellulase and BSA were independently incubated with filter paper for 0, 6, 12 or 24 h. Proteins in the supernatants (i.e., free proteins) or filter paper (i.e., bound proteins) of each incubation mixture were analyzed by SDS-PAGE. When cellulase was incubated alone with filter paper, the major protein band of supernatant of cellulase, shown at 75 kDa, markedly decreased with increasing incubation time (FIG. 9-a). There is possibility that the weakening of cellulase band could be caused the degradation of the cellulase by protease possibly contained in the cellulase preparation. But the bound cellulase band at 75 kDa significantly increased with time as seen in FIGS. 9 (e) and (f). Therefore, the effect of protein degradation by protease possible contained in the cellulase preparation was thought to be insignificant. In contrast, the band strength of the filter paper-derived cellulase increased with increasing incubation time (FIG. 9-e). These results strongly confirm the binding of cellulase to the filter paper. When BSA, a control protein, was incubated with filter paper, there was no significant change in band strengths of cellulase in either the supernatant or the filer paper (FIG. 9-d and h). This confirms the insignificant binding of BSA to pure cellulose as previously reported (Rudolf et al. 2008; Yang et al. 2006). These behaviors of cellulase against filter paper were similar when cellulase was incubated along with BsEXLX1 (FIGS. 9-b and 9-f).

BsEXLX1 also showed strong binding activity to the filter paper when incubated alone or with cellulase. The band strength of BsEXLX1 from the supernatant became weaker with time (FIGS. 9-b and c), but that from the filter paper increased with time (FIGS. 9-f and g). Thus, as mentioned earlier, a less distinct or decreased level of synergism by BsEXLX1, which was observed when the concentrations of cellulase and BsEXLX1 were increased to certain levels, might be a result of the increased competition of the proteins for a fixed amount of binding sites in cellulose substrate.

### EXAMPLE 8

### Effect of Temperature on Synergism in Cellulose Hydrolysis

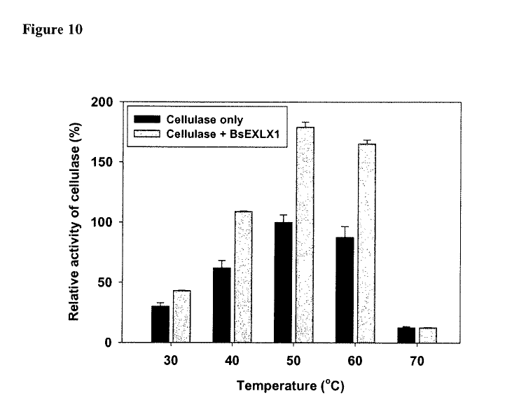

The cellulose hydrolysis reaction temperature was set at 50° C., the optimal temperature of cellulase recommended by the manufacturer. The sugar yield from filter paper incubated with cellulase alone or with both cellulase and BsEXLX1 was greatest at 50° C. (FIG. 10), which decreased with a further increase in reaction temperature. The reaction yield was markedly lower at 70° C. than at other temperatures, possibly due to thermal denaturation of the enzymes. Thus, the synergistically increased cellulase activity due to BsEXLX1 was parallel with the activity of cellulase alone in terms of its dependence on the reaction temperature.

### EXAMPLE 9

### Effect of pH on Synergism in Cellulose Hydrolysis

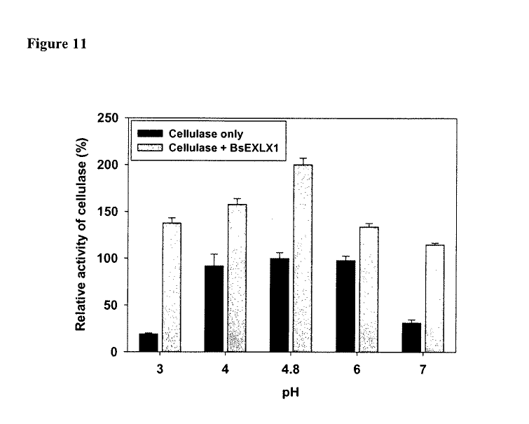

All of the above hydrolysis reactions were conducted at pH 4.8, the optimal pH of the cellulase recommended by its manufacturer. We investigated whether the optimal pH for maximal hydrolysis yield was maintained when BsEXLX1 was added. We tested a pH range of pH 3-7 and found that the activity of cellulase (without BsEXLX1) remained almost constant at pHs of 4, 4.8 and 5 (FIG. 11), indicating that cellulase activity did not significantly differ with pH in the range of pH 4-6. However, when BsEXLX1 was added, the cellulase activity was much more influenced by pH. This may be related to the higher dependency of BsEXLX1 on pH than cellulase.

### EXAMPLE 10

### Tensile Strength of Filter Paper Treated with BsEXLX1

To find possible filter paper-weakening activity of BsEXLX1, the tensile strength of BsEXLX1-treated filter papers was measured by a Universal Testing Machine (UTM; Instron, Norwood, Mass., USA). Whatman No. 3 filter paper was cut into strips of 2×5.5 cm and placed between clamps of the UTM. The clamped filter paper strips were incubated in 10 mL of sodium acetate buffer (50 mM, pH 4.8) containing 600 g/ml BsEXLX1 or BSA as a protein control. For negative controls, buffer containing BSA at the same concentration as that of BsEXLX1 or buffer alone was used as a bathing solution for the filter paper. For a positive control, an 8 M urea solution was used. The incubated paper strips were extended by applying a load of 5 g to the lower clamp for 10 min, where the crosshead speed was 0.5 mm/min. The maximum force (Fₘₐₓ) was measured, and the tensile strength (rₘₐₓ) was calculated from the equation rₘₐₓ=Fₘₐₓ/A, where A=cross sectional area and Fₘₐₓ=maximum load (kg). The experiments were done in triplicate and expressed as the means±standard deviations.

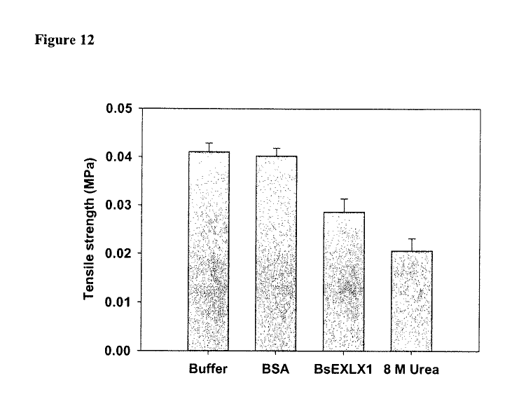

To test any possible disruption effect of BsEXLX1 on cellulose, filter paper strips were incubated in a buffer containing purified BsEXLX1, BSA (a negative control), buffer alone (a negative control) or an 8 M urea solution (a positive control) for 1 h. As seen in FIG. 12, there was no significant difference in tensile strength between paper strips bathed in buffer and buffer containing BSA. The positive control experiment with an 8 M urea solution resulted in 50% reduction of tensile strength compared to buffer alone. Paper strips incubated in the buffer containing BsEXLX1 showed 29% reduction of tensile strength compared to that of strips in buffer alone. The filter paper-weakening activity revealed by the tensile tests indicated that BsEXLX1 also has disruption activity against cellulose similarly to that of swollenin (Baker et al. 2000; Saloheimo et al. 2002) and plant expansins (Cosgrove 2000a; Cosgrove 2000b; McQueen-Mason and Cosgrove 1994)

### EXAMPLE 11

### SEM of Filter Paper Treated with a Bacterial Expansin, BsEXLX1

Scanning electron microscopy (SEM) was conducted to analyze microstructural changes of filter paper treated with BsEXLX1. Three different filter paper samples such as filter paper incubated in a buffer solution only, incubated with BsEXLX1 and incubated in an 8 M urea solution were prepared under the same conditions as in the measurement of tensile strength of filter paper. Prior to analysis by SEM, filter paper samples were dried in a vacuum drying-oven at 45° C. for 1 day, after which they were coated with gold-palladium. Photomicrographs of the samples were then taken using a scanning electron microscope (Hitachi S-4700, Tokyo, Japan) at a voltage of 10 kV.

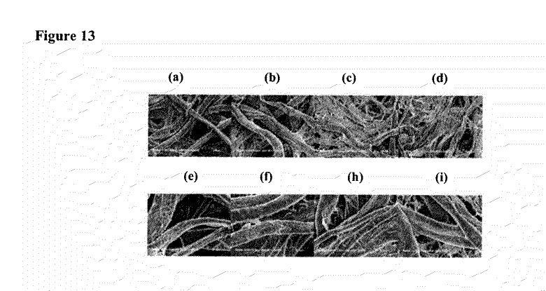

The microphotographs of the filter paper samples were taken by SEM as seen in FIG. 10. SEM revealed differences in microstructures of filter paper samples incubated in buffer solutions containing different substances. Filter paper incubated in an 8 M urea solution as a positive control as shown in FIGS. 13 (b) and (f) showed that the fibrils of filter paper had less interconnected and overlapped shape probably due to the disruptive activity of urea. Therefore, the tensile strength decrease after incubation in 8 M urea as seen in FIG. 9 was in good agreement with the ultrastructural changes revealed by SEM. The similar structural shape change was exhibited in the filter paper incubated in the buffer solution containing BsEXLX1 as in FIGS. 13 (a) and (e). These results also correlate with the reduced tensile strength in the BsEXLX1-treated filter paper in FIG. 12. Meanwhile, the negative controls such as the filter paper samples incubated in the buffer solution only (FIGS. 13 (c) and (h)) or the buffer solution containing BSA (FIGS. 13 (d) and (i)) exhibited that fibrils looked much more dense, overlapped and interconnected each other. Therefore, these SEM results of the filter paper samples incubated in the buffer solution alone and the BSA-containing buffer solution well agreed with the tensile strength data in FIG. 12.

We examined and validated the predicted molecular functions of BsEXLX1, a 24 kDa protein from B. subtilis, which has a structural similarity to a β-expansin from maize. The recombinant protein was found to also have functional homology to plant expansins. BsEXLX1 exhibited both cellulose-binding and cellulose-weakening activities towards filter paper, similarly to plant expansins. Furthermore, BsEXLX1 also demonstrated a significant synergism when added to a cellulose hydrolysis reaction mixture containing low-dose cellulase and filter paper in reaction buffer. At 0.012 FPU of cellulase and 300 μg of BsEXLX1, the sugar yield due to the synergistic effect of BsEXLX1 was 5.7-fold greater than that obtained by cellulase only. The filter paper-weakening activity by BsEXLX1 quantified as tensile strength measurement was visually confirmed by SEM.

These results lead to the conclusion that the prokaryotic expansin protein of the present invention performs the same function (i.e. cellulose expansion activity) as existing plant expansin proteins and can be produced at greatly reduced cost on an industrial scale, unlike plant expansin proteins. Particularly, when lignocellulosic biomass is hydrolyzed into glucose using the prokaryotic expansin protein of the present invention together with cellulase, the hydrolysis rate of cellulose can be markedly increased by the cellulase, resulting in improved yield of the glucose. In actuality, the use of the prokaryotic expansin according to the present invention enables the production of bioenergy at low cost with a reduced amount of enzyme used. In addition, the prokaryotic expansin protein of the present invention softens and varies the textures of pulp, cotton fibers (e.g., jeans), etc. Therefore, the prokaryotic expansin protein of the present invention can be used for various purposes, such as biopulping and biostoning.

### Keywords

cellulase; cellulose hydrolysis; synergism; bacterial expansin; biofuel; structural homolog

### REFERENCES

Adney B, Baker J. 1996. Measurement of cellulase activities, Chemical Analysis and Testing Task: LAP-006. National Renewable Energy Laboratory, Golden, Colo., USA.
Aslanidis C, Dejong P J. 1990. Ligation-independent cloning of PCR products (LIC-PCR). Nucleic Acids Res. 18(20):6069-6074.
Baker J O, King M R, Adney W S, Decker S R, Vinzant T B, Lantz S E, Nieves R E, Thomas S R, Li L C, Cosgrove D J and others. 2000. Investigation of the cell-wall loosening protein expansin as a possible additive in the enzymatic saccharification of lignocellulosic biomass. Appl. Biochem. Biotechnol. 84-6:217-223.
Berlin A, Maximenko V, Bura R, Kang K Y, Gilkes N, Saddler J. 2006. A rapid microassay to evaluate enzymatic hydrolysis of lignocellulosic substrates. Biotechnol. Bioeng. 93(5): 880-886.
Boraston A B, Kwan E, Chiu P, Warren R A J, Kilburn D G. 2003. Recognition and hydrolysis of noncrystalline cellulose. J. Biol. Chem. 278(8):6120-6127.
Canard G, Koivula A, Soderlund H, Beguin P. 2000. Cellulose-binding domains promote hydrolysis of different sites on crystalline cellulose. Proc. Natl. Acad. Sci. U.S.A 97(19):10342-10347.
Cosgrove D J. 2000a. Loosening of plant cell walls by expansins. Nature 407(6802):321-326.
Cosgrove D J. 2000b. New genes and new biological roles for expansins. Curr. Opin. Plant Biol. 3(1):73-78.
Cosgrove D J. 2001. Enhancement of accessibility of cellulose by expansins. U.S. Pat. No. 6,326,470 B1.
Cosgrove D J. 2007. Use of GR2 proteins to modify cellulosic material and to enhance enzymatic and chemical modification of cellulose. US Patent 2007/0166805 A1.
Graslund S, Nordlund P, Weigelt J, Bray J, Gileadi O, Knapp S, Oppermann U, Arrowsmith C, Hui R, Ming J and others. 2008. Protein production and purification. Nat. Methods 5:135-146.
Gregg D J, Boussaid A, Saddler J N. 1998. Techno-economic evaluations of a generic wood-to-ethanol process: Effect of increased cellulose yields and enzyme recycle. Bioresour. Technol. 63(1):7-12.
Han Y J, Chen H Z. 2007. Synergism between corn stover protein and cellulase. Enzyme Microb. Technol. 41(5):638-645.
Holm L, Park J. 2000. DaliLite workbench for protein structure comparison. Bioinformatics 16(6):566-567.
Irwin D C, Spezio M, Walker L P, Wilson D B. 1993. Activity studies of eight purified cellulases—specificity, synergism, and binding domain effects. Biotechnol. Bioeng. 42(8):1002-1013.
Kende H, Bradford K J, Brummell D A, Cho H T, Cosgrove D J, Fleming A J, Gehring C, Lee Y, McQueen-Mason S, Rose J K C and others. 2004. Nomenclature for members of the expansin superfamily of genes and proteins. Plant Mol. Biol. 55(3):311-314.
Kim K H, Tucker M, Nguyen Q. 2005. Conversion of bark-rich biomass mixture into fermentable sugar by two-stage dilute acid-catalyzed hydrolysis. Bioresour. Technol. 96(11):1249-1255.
Lynd L R, Laser M S, Bransby D, Dale B E, Davision B, Hamilton R, Himmel M, Keller H, McMillan J M, Sheehan J and others. 2008. How biotech can transform biofuels. Nat. Biotechnol. 26(2):169-172.
McQueen-Mason S, Cosgrove D J. 1994. Disruption of hydrogen-bonding between plant-cell wall polymers by proteins that wall extension. Proc. Natl. Acad. Sci. U.S.A. 91(14):6574-6578.
Medve J, Stahlberg J, Tjerneld F. 1994. Adsorption and synergism of cellobiohydrolase-I and cellohydrolase-II of Trichoderma-reesei during hydrolysis of microcrystalline cellulose. Biotechnol. Bioeng. 44(9):1064-1073.
Merino S T, Cherry J. 2007. Progress and challenges in enzyme development for Biomass utilization. Adv. Biochem. Eng. Biotechnol. 108: 95-120.
Moser F, Irwin D, Shaolin Chen D B W. 2008. Regulation and characterization of Thermobifida fusca carbohydrate-binding module proteins E7 and E8. Biotechnol. Bioeng. 100(6):1066-1077.
Nguyen Q A, Tucker M P, Boynton B L, Keller F A, Schell D J. 1998. Dilute acid pretreatment of softwoods. Appl. Biochem. Biotechnol. 70-72:77-87.
Petrella S, Herman R, Sauvage E, Filee P, Joris B, Charlier P. 2006. Crystal Structure of a sement derivatiove of expa from Bacillus subtilis at 2.5 angstrom. http://dx.doi.org/10.2210/pdb2bh0/pdb.
Pettersen E F, Goddard T D, Huang C C, Couch G S, Greenblatt D M, Meng E C, Ferrin T E. 2004. UCSF chimera—A visualization system for exploratory research and analysis. J. Comput. Chem. 25(13):1605-1612.
Qin L, Kudla U, Roze E H A, Goverse A, Popeijus H, Nieuwland J, Overmars H, Jones J T, Schots A, Smant G and others. 2004. Plant degradation: A nematode expansin acting on plants. Nature 427(6969):30-30.
Rudolf A, Baudel H, Zacchi G, Hahn-Hagerdal B, Liden G. 2008. Simultaneous saccharification and fermentation of steam-pretreated bagasse using Saccharomyces cerevisiae TMB3400 and Pichia stipitis CBS6054. Biotechnol. Bioeng. 99:783-790.
Saloheimo M, Paloheimo M, Hakola S, Pere J, Swanson B, Nyyssonen E, Bhatia A, Ward M, Penttila M. 2002. Swollenin, a Trichoderma reesei protein with sequence similarity to the plant expansins, exhibits disruption activity on cellulosic materials. Eur. J. Biochem. 269(17):4202-4211.
Sampedro J, Cosgrove D J. 2005. The expansin superfamily. Genome Biol. 6(12).
Sampedro J, Carey R E, Cosgrove D J. 2006. Genome histories clarify evolution of the expansin superfamily: new insights from the poplar genome and pine ESTs. J. Plant Res. 119:11-21
Sherrad E C, Kressman F W. 1945. Review of processes in the United States prior to World War II. Ind. Eng. Chem. 37(1):5-8.
Torget R, Himmel M E, Grohmann K. 1991. Dilute sulfuric acid pretreatment of hardwood bark. Bioresour. Technol. 35(3):239-246.
Wingren A, Galbe M, Zacchi G. 2003. Techno-economic evaluation of producing ethanol from softwood: Comparison of SSF and SHF and identification of bottlenecks. Biotechnol. Prog. 19(4):1109-1117.
Woodward J, Lima M, Lee N E. 1988. The role of cellulase concentration in determining the degree of synergism in the hydrolysis of microcrystalline cellulose. Biochem. J. 255(3):895-899.
Xiao Z Z, Storms R, Tsang A. 2004. Microplate-based filter paper assay to measure total cellulase activity. Biotechnol. Bioeng. 88(7):832-837.
Yang B, Willies D M, Wyman C E. 2006. Changes in the enzymatic hydrolysis rate of Avicel cellulose with conversion. Biotechnol. Bioeng. 94(6):1122-1128.
Yennawar N H, Li L C, Dudzinski D M, Tabuchi A, Cosgrove D J. 2006. Crystal structure and activities of EXPB1 (Zea m 1), a b-expansin and group-1 pollen allergen from maize. Proc. Natl. Acad. Sci. U.S.A. 103(40):14664-14671.

What is claimed is:

1. A prokaryotic expansin protein for activating the expansion of cellulose wherein the prokaryotic expansin protein has cellulose expansion activity.

2. The prokaryotic expansin protein of claim 1, wherein the prokaryote is selected from the group consisting of Bacillus subtilis, Hahella chejuensis (KCTC 2396), Dictyostelium discoideum, Neosartorya fischeri, Aspergillus fumigatus, Aspergillus clavatus, Aspergillus oryzae, Aspergillus terreus, Penicillium chrysogenum, Aspergillus niger, Emericella nidulans, Magnaporthe grisea, Pyrenophora tritici-repentis, Phaeosphaeria nodorum, Sclerotinia sclerotiorum, Frankia sp., Streptomyces sviceus, Sorangium cellulosum, Stigmatella aurantiaca, Plesiocystis pacifica, Myxococcus xanthus, Leptothrix cholodnii, Roseiflexus sp., Roseiflexus castenholzii, Chloroflexus aurantiacus, Herpetosiphon aurantiacus, Acidovorax avenae subsp, Pectobacterium atrosepticum, Bacillus licheniformis, Xanthomonas campestris pv. campestris, Bacillus pumilus, Xanthomonas oryzae pv. oryzae, Ralstonia solanacearum, Clavibacter michiganensis, Xylella fastidiosa, Nakamurella multipartite, Micromonospora sp., Catenulispora acidiphila and Dickeya zeae.

3. The prokaryotic expansin protein of claim 2, wherein the prokaryote is Bacillus subtilis, Stigmatella aurantiaca, Xanthomonas oryzae or Hahella chejuensis.

4. The prokaryotic expansin protein of claim 1, wherein the expansin protein comprises an amino acid sequence selected from the group consisting of the amino acid sequences set forth in SEQ ID NOS: 1-47.

5. The prokaryotic expansin protein of claim 4, wherein the expansin protein is the amino acid sequence set forth in SEQ ID NO: 20, 22, 30 or 35.

6. A cellulose-degrading composition comprising the prokaryotic expansin protein of claim 1.

7. The cellulose-degrading composition of claim 6, further comprising a cellulose-degrading enzyme.

8. The cellulose-degrading composition of claim 7, wherein the cellulose-degrading enzyme is selected from the group consisting of cellulase, cellobiohydrolase, endoglucanase, cellobiase, and mixtures thereof.

9. The cellulose-degrading composition of claim 7, wherein the cellulose-degrading composition comprises 0.01 to 0.05 FPU of the cellulose-degrading enzyme and 200 to 400 μg of the prokaryotic expansin protein per g cellulose.

10. A method for degrading cellulose, comprising reacting the cellulose-degrading composition of claim 6 with the cellulose at 40 to 60° C.

11. A method for degrading cellulose, comprising reacting the cellulose-degrading composition of claim 6 with the cellulose at a pH not higher than 7.

12. A prokaryotic expansin protein having a homology of at least 60% to an amino acid sequence of an expansin (EXLX1) originating from Hahella chejuensis and having cellulose expansion activity.

13. A method for producing bioenergy, comprising treating lignocellulosic biomass with the cellulose-degrading composition of claim 6 to degrade cellulose contained in the lignocellulosic biomass and eventually to produce reducing sugar.

14. A method for softening paper or pulp, comprising treating the paper or pulp with the cellulose-degrading composition of claim 6 to degrade cellulose contained in the paper or pulp.

15. A method for softening a fiber or fabric, comprising treating the fiber or fabric with the cellulose-degrading composition of claim 6 to degrade cellulose contained in the fiber or fabric.

16. A method for producing a prokaryotic expansin protein on an industrial scale, the method comprising
a) finding a prokaryotic protein having a structural similarity to a plant expansin,
b) cloning the prokaryotic protein, and
c) expressing the cloned prokaryotic protein in a strain.

17. The method of claim 16, wherein the plant expansin comprises the amino acid sequence of SEQ ID NO: 57.
